(19) United States
(12) Patent Application Publication
(10) Pub. No.: US 2008/0019975 A1
(43) Pub. Date: Jan. 24, 2008

(54) Novel Regimens for Treating Diseases and Disorders

(75) Inventor: James R. Gorman, Natick, MA (US)

Correspondence Address: FISH & RICHARDSON P.C., PO BOX 1022, MINNEAPOLIS, MN, 55440-1022, US

(73) Assignee: BioAssets Development Corporation

(21) Appl. No.: 11/775,206

(22) Filed: Jul. 9, 2007

Related U.S. Application Data
(60) Provisional application No. 60/847,493, filed on Sep. 27, 2006.
(60) Provisional application No. 60/819,555, filed on Jul. 7, 2006.

(51) Int. Cl. A61K 39/395; A61K 31/192; A61K 31/4168; A61K 31/4402; A61P 19/00; A61P 29/00; A61P 9/00; A61P 37/00; A61P 25/00; A61K 31/70; A61K 31/7105; A61K 38/02

(52) U.S. Cl. 424/145.1; 424/130.1; 424/158.1; 514/2; 514/352; 514/401; 514/44; 514/54; 514/570

(57) ABSTRACT

Methods and materials are provided for induction-maintenance regimens of targeted anti-inflammatory therapies (TATs) for treatment of a variety of diseases and disorders. Preferred embodiments include administration of one or more TATs using an induction regimen comprising a lower dose per administration administered by a more invasive and/or more localized route, followed by administration of one or more TATS using a maintenance regimen, comprising a higher dose per administration administered by a less invasive and/or less localized route.

### CROSS-REFERENCE TO RELATED APPLICATIONS

This application claims priority under 35 U.S.C. §119 to U.S. Provisional Patent Applications 60/819,555, filed Jul. 7, 2006 and 60/847,493, filed Sep. 27, 2006; the entire contents of which are incorporated by reference herein in their entirety.

This application is related to U.S. application Ser. No. ______ (Attorney Docket No. 21782-005001) and Ser. No. ______ (Attorney Docket No. 21782-006001), both filed concurrently herewith on Jul. 9, 2007, the entire contents of which are incorporated by reference herein in their entirety.

### FIELD OF THE INVENTION

The present disclosure relates to materials and methods for treating diseases and disorders, including diseases and disorders in which inflammatory cytokines (ICs) and inflammatory mediators (IMs) are implicated as causing, contributing to, or perpetuating the pathophysiology of the disease or disorder. More particularly, this disclosure relates to the use of a targeted anti-inflammatory therapy (TAT), such as an IC inhibitor (IC-I) or an IM inhibitor (IM-I), including tumor necrosis factor-α (TNF) inhibitors (TNF-Is), administered by novel induction and maintenance regimens as described herein, to treat subjects.

### BACKGROUND OF THE INVENTION

Role of ICs and IMs in Diseases and Disorders

ICs and/or IMs are implicated as causing, contributing to, exacerbating, or perpetuating the pathophysiology of a wide range of prevalent and troublesome diseases and disorders. New classes of TATs, including protein therapeutics, offer new possibilities of targeted therapy, but also have inherent limitations in their usage in certain disorders. For example, protein TATs such as TNF-Is do not readily access certain tissues. Invasive administration is limited by risk, expense, and availability, and its use for protein drugs is new or untried. To address some of these limitations, the inventor describes novel regimens by which TATs can be administered, to enhance their use and efficacy in many disorders, including spinal disorders.

A wide variety of inducers can cause inflammation in the body, including trauma, injury, disease, surgery, infection and cytokines. Such stimuli can induce the production of IC by a wide variety of cells, including cells of the immune system, cells of the central and peripheral nervous systems and cells from other tissues and organs (FIG. 1). Certain IC, such as TNF, IL-1, IL-6, IL-8, IL-12, IL-15, IL-17, IL-18, IL-23, IFN-γ, GM-CSF, and MCP-1, play key roles in the induction and maintenance of inflammation. A subset of cytokines called chemokines, such as IL-8 and MCP-1, function in concert with other IC during inflammation to recruit cells from the blood or cerebrospinal fluid to the site of injury. A wide variety of cell types comprise the inflammatory cell infiltrate (FIG. 1). Cells recruited to the site of injury, particularly monocytes, macrophages and dendritic cells, produce additional IC which collectively modulate cell maturation, proliferation, activation and angiogenesis. These IC act on both infiltrating cells and local tissue cells to produce and release inflammatory mediators (IM). Key IM include nitric oxide (NO), produced via activation of inducible NO synthase (iNOS), prostaglandinE2 (PGE2), an arachidonic acid metabolite resulting from the induction of the COX-2 enzyme, the matrix metalloproteinases (MMPs) MMP-1 (collagenase-1), MMP-2 (gelatinase A), MMP-3 (stromelysin), MMP-7 (matrilysin), MMP-9 (gelatinase B) and MMP-13 (collagenase-3), and the matrix-degrading aggrecanases ADAMTS4 and ADAMTS5 of the Adamalysin family of proteases. As illustrated in FIG. 1, IC and IM act individually and in concert to cause inflammation and tissue damage, for example in irritation, inflammation, and injury of the spinal nerve root (NR). They also cause degradation of proteoglycans and extracellular matrix, as in matrix destruction in intervertebral disks and cartilage.

Elevated levels of the ICs and IMs discussed above, including in particular TNF, play an important role in pathologic inflammation and have been implicated in the pathophysiology of a variety of human diseases and disorders, including pain, spinal disorders, orthopedic disorders, inflammatory diseases, immune system disease, metabolic disorders, cardiovascular disease and diseases of endothelial dysfunction, and disorders of the central and peripheral nervous systems. Other diseases and disorders where ICs and IMs, and in particular TNF, may play a role are malignancy, anemia, hepatic disorders (including HBV and HCV infection, autoimmune hepatitis, fatty liver disease, hepatotoxicity, liver failure, non-alcoholic hepatitis, alcoholic hepatitis, fibrosis), nail disease, endometriosis, prostatitis, scar tissue formation, periodontal disease, spinal cord edema, pancreatitis, and gout.

Novel Regimens of TATs, Including TNF-Is, in the Treatment of Spinal Disorders

Spinal disorders such as herniated disk (HD) cause mechanical compression of spinal nerve roots (NRs) and nerves, initiating a biochemical cascade in which ICs such as TNF play an essential role. The resulting NR injury can cause radiating pain along the distribution of the affected NR, colloquially known as “sciatica” when occurring in the lower back and extending (“radiating”) into the buttock, thigh, or leg, in the distribution of the sciatic nerve. TNF and other ICs and IMs are increasingly implicated in controlling the pathophysiology of NR injury, inflammation and pain, in the destructive process of degenerative disk disease (DDD), and in other spinal disorders.

Severe or persistent radicular pain is frequently associated with HD. Patients diagnosed with HD may receive an initial trial of conservative therapy including rest and oral analgesics, and conventional anti-inflammatory therapy, such as non-steroidal anti-inflammatory drugs (NSAIDs) and oral glucocorticoids. When relief provided by conservative therapy proves inadequate, treatment typically progresses to opioid analgesics and to more invasive, expensive epidural injections of steroids or of local anesthetics (LAs). If these measures fail, the patient will often undergo a spinal surgery procedure, for example a disk removal, or for many conditions, implantation of a spinal device such as an artificial disk, or fusion of the adjacent vertebrae.

TATs such as TNF-Is are not routinely used in current treatment of spinal disorders such as HD. For example, the currently marketed TNF-Is Enbrel® (etanercept), Humira® (adalimumab), and Remicade® (infliximab) are not routinely prescribed to spine patients. The potential efficacy of intravenous (IV) or subcutaneous (SC) administration of TNF-Is has been tested in preliminary human clinical trials in patients with HD and sciatic pain with mixed results. The only published blinded clinical trial used IV infliximab in sciatica patients and failed to show a difference between patients treated with the drug versus those treated with placebo saline infusion. As described below, current practice and teaching poses specific barriers to use of TNF-Is in patients found eligible for spinal surgery, and additional barriers in patients who actually undergo spinal surgery.

First, the currently marketed TNF-I compounds, Enbrel® (etanercept), Humira® (adalimumab), and Remicade® (infliximab), are protein therapeutics, either monoclonal antibodies or soluble cytokine receptor fusion proteins. Enbrel®, Humira®, and Remicade® are approved for use by systemic routes of administration, either IV or SC. Such agents are widely viewed as not crossing the blood brain barrier, and therefore of limited use in treating disorders of the spinal NR such as HD or SS. The disk itself is poorly vascularized and is not well accessible to protein therapeutics administered by parenteral routes. There is little or no literature to guide the use of emerging TATs such as protein therapeutics by invasive or localized routes of administration such as epidural or intradiskal administration. At the same time, invasive routes of administration carry risk, expense, restricted availability to patients. Therapies for which the efficacy and/or safety is limited to an invasive route of administration are inherently limited in their availability, cost and therefore utility. Thus, available TATs such as the TNF-Is have not been adapted for treatment of spinal disorders such as HD.

Second, treatment with the marketed TNF-Is has been linked with an increased risk of certain infections, a risk of significant potential concern to in invasive procedures such as spinal surgery. This perceived potential for increased risk of infection presents a barrier to TNF-I use in patients eligible for or scheduled for spine surgery. Thus, current perceptions of TNF-Is and current practice in management of perceived infection risk further limit use of TNF-Is and other emerging TATs in patients found eligible for a spinal surgery procedure. Similarly, once a determination is made that the patient will actually undergo the procedure, TNF-Is and other TATs are not prescribed. The spinal surgery procedure is viewed as likely to alleviate the mechanical disorder. The inventor has observed that even when a disk or lamina is removed, the removal procedure itself can further exacerbate the disorder, likely through activation of pathways that release ICs and IMs. Thus, patients undergoing a spinal surgery procedure are, surprisingly, likely to benefit from an administration of a TAT such as a TNF-I, through improved outcome of the spinal surgery procedure.

In summary, many patients with a spinal disorder such as HD would benefit from therapy with a TAT, if the agent could be delivered to the site of the pathology, such as the inflamed NR, by a route that is safe, effective, and readily available. However, limitations of the agents, including infection risk and disappointing efficacy when delivered systemically, poor penetration into the nervous system, lack of prior use in invasive routes of administration, and high cost and limited availability of invasive administration approaches, limit their use. Induction-maintenance TNF-I regimens for inflammatory disorders such as Crohn's disease have comprised an induction phase with systemically administered loading doses many times higher per administration than the maintenance regimen dose per administration, which is delivered systemically by the same route as the loading dose (see U.S. Pat. Publ. 2006/0009385). The inventor has discovered that for many diseases including spinal disorders with radiating pain, a preferred regimen starts with a smaller dose, targeted locally and/or invasively, followed by a larger dose, delivered by a less invasive, less localized route.

The efficacy and suitability of these agents for this class of patients is surprising. Contrary to current practice, when administered by novel regimens, many patients for whom TATs would not currently be prescribed could benefit from treatment with a TAT such as a TNF-I. For example, patients with spinal disorders such as HD and sciatica could be treated early in the course of the disorder, before they become eligible for surgery. Further, if the patient becomes eligible to undergo spinal surgery, treatment with a TAT using the novel regimens described herein may allow the subject to postpone or avoid the need for surgery through practice of the invention. For patients who do undergo a spinal surgery procedure, TAT therapy administered by the regimens described can improve the outcome and speed post-operative recovery.

Similarly, for diverse non-spinal disorders, tailored novel regimens for TAT administration can be surprisingly effective and safe. These novel regimens incorporate an induction phase using a lower dose per administration delivered more invasively and/or more locally, followed by a maintenance phase using a larger dose per administration delivered by a less invasive and/or less local route. Optionally, they may include peri-operative interruption.

### SUMMARY OF THE INVENTION

The present inventor has discovered novel therapeutic regimens using TATs that are particularly effective for the treatment of pain and other diseases and disorders. The novel therapeutic regimens of the present invention are particularly effective for the treatment of pain, inflammatory diseases, spinal disorders, immune system disease, diabetes, cardiovascular disease, and disorders of the central and peripheral nervous system.

In one embodiment, herein is disclosed a method for treating a disease or disorder. The methods includes administering to a subject in need thereof an induction regimen of a direct TNF-I and a maintenance regimen of a direct TNF-I, where the TNF-I of the induction regimen is administered at a lower dose per administration than the dose per administration of the TNF-I of the maintenance regimen, and where the TNF-I of the induction regimen is administered more locally and/or in a more invasive manner than the TNF-I of the maintenance regimen.

In an alternative embodiment, the method includes administering to a subject in need thereof an induction regimen of an NFκB-I, and a maintenance regimen of an NFκB-I, where the NFκB-I of the induction regimen is administered at a lower dose per administration than the dose per administration of the NFκB-I of the maintenance regimen, and where the NFκB-I of the induction regimen is administered more locally and/or in a more invasive manner than the NFκB-I of the maintenance regimen.

In one embodiment, the disease or disorder to be treated by these methods includes a pain syndrome, a spinal disorder, an orthopedic disorder, an inflammatory disease, an immune system disease, a metabolic disorder, a cardiovascular disease, a disease of endothelial dysfunction, a disorder of the central nervous system, and a disorder of the peripheral nervous system. Pain syndromes that may be treated using the methods disclosed herein may be selected from the following group: acute pain, chronic pain, complex regional pain syndrome type I, complex regional pain syndrome type II, neuropathic pain, post-operative pain, pain caused by inflammation, chronic lower back pain, sciatica, cluster headaches, post-herpetic neuralgia, phantom limb pain, stump pain, central pain, dental pain, opioid-resistant pain, visceral pain, surgical pain, bone injury pain, pain during labor and delivery, pain resulting from burn, post partum pain, migraine, angina pain, fibromyalgia, and genitourinary tract-related pain, including cystitis, and nociceptive pain. Spinal disorders that may be treated using the methods disclosed herein may be selected from the following group: disk disorders, including HD and DDD, disorders of spinal stability, disorders of the vertebrae including kyphosis and facet joint disease, nerve disorders, SS, arthritic spinal disorders, back pain conditions, and failed back surgery syndrome (FBSS). In one aspect, a disk disorder may be a herniated disk or a degenerative disk disorder. In a further aspect, a disk disorder may be selected from the following group: prolapsed disk, protruding disk, extruded disk, bulging disk, sequestered disk, DDD, DDD with internal disk derangement, diskogenic pain, annular disorder, annular bulge, annular tear, nucleus pulposus degeneration, NR compression, radicular pain, radiculopathy, sciatica, radiating pain, and distraction injury. A disorder of spinal stability may be selected from the following group: spondylolysis, spondylolisthesis, lytic spondylolisthesis, degenerative spondylolisthesis, lumbar spondylolisthesis, isthmic spondylolisthesis, and grade 1 spondylolisthesis. Vertebral disorders that may be treated using the methods disclosed herein may be selected from the following group: vertebral body collapse, vertebral body degeneration, vertebral body compression, metastasis, kyphosis, facet joint disease, facet disease, facet joint disease facet joint syndrome, and impinging facet joints. Arthritic spinal disorders that may be treated using the methods disclosed herein may be selected from the following group: rheumatoid arthritis, ankylosing spondylitis, osteoarthritis, degenerative spinal arthritis, cervical arthritis, thoracic arthritis, DDD, bone spurs, osteophytes, and an arthritic facet joint disorder. Nerve disorders that may be treated using the methods disclosed herein may be selected from the following group: nerve compression syndrome, NR compression, NR irritation, NR inflammation, nerve entrapment, nerve compression by a tumor, lumbago, HD, SS, neural foraminal narrowing, pinched nerve, and sciatica. Back pain conditions that may be treated using the methods disclosed herein may be selected from the following group: back pain, low back pain, chronic back pain, radicular pain, radiating pain, sciatica, radiculitis, lumbar radiculopathy, diskogenic pain, facet pain, cervical radiculopathy, cervical headache, whiplash, whiplash headache, whiplash associated disorder, scoliosis, scoliosis pain, post-operative pain, post-operative leg pain, and fibromyalgia. Orthopedic disorder that may be treated using the methods disclosed herein may be selected from the following group: an orthopedic joint disorder of the hip, knee, shoulder, ankle, elbow, wrist, toe, finger, sacro-iliac, and spinal facet joint. Inflammatory disorders that may be treated using the methods disclosed herein may be selected from the following group: chronic inflammatory airway disorders (including asthma, alergic asthma, non-allergic, intrinsic asthma, exercise-induced asthma, nocturnal asthma, occupational asthma, steroid resistant asthma, exercise-induced bronchospasm, and chronic obstructive pulmonary disease); chronic inflammatory bowel diseases (including ulcerative colitis, and Crohn's disease); chronic inflammatory connective tissue diseases (including lupus erythematosus, scleroderma, Sjogren's syndrome, poly- and dermatomyositis, vasculitis, and MCTD); chronic inflammatory joint diseases (including rheumatoid arthritis juvenile chronic arthritis, Still's disease, rheumatoid spondylitis, lupus erythematosus, ankylosing spondylitis, psoriatic arthritis, and reactive arthritis, rheumatoid arthritis of the hip, bursitis of the hip, and osteoarthritis of the hip); chronic inflammatory skin diseases (including psoriasis, diskoid lupus erythematosus, scleroderma, hives, rosacea, dermatitis, and atopic dermatitis); spondyloarthropies; cardiomyopathy; atherosclerosis vasculitis (including anti-neutrophil cytoplasmic Ab (ANCA)-associated vasculitis and chronic and relapsing ANCA-associated vasculitis); acute renal disease; chronic renal disease; glomerulonephritis; inflammatory eye disorders (including retinitis); tuberculosis; chronic cholecystitis; bronchiectasis; Hashimoto's thyroidiitis; Silicosi; pneumoconioses; hyper-IgG4 disease; ileus; inflammatory side effects associated with a pharmaceutical agent; and post operative inflammation.

In one embodiment, treatment is administered peri-operatively to a surgery of the subject, where the surgery is an orthopedic surgery selected from the following group: surgery to the hand, elbow, shoulder, spine, hip, knee, or ankle joint, arthroscopy (including of the wrist, elbow, shoulder, spine, hip, knee, ankle, or any other joint); carpal tunnel release; knee arthroscopy (including with meniscectomy, chondroplasty or anterior cruciate ligament reconstruction); tendon repair or replacement (including rotator cuff tendon repair); ligament repair or replacement; fracture repair; and bone graft.

In an embodiment, the surgery involves the implantation revision, or removal of an orthopedic device used for replacement or repair of a joint structure of the hand, foot, wrist, elbow, shoulder, spine, hip, knee, or ankle joint. In one aspect, a device is selected from the following group: a stent; a pump; an annular repair device; a nucleus replacement device; a dynamic stabilization device; a synthetic bone graft substitute; an allograft cage; a motion preservation device; a pedicle screw; a facet screw; a vertebral body replacement; a hip replacement device; a knee replacement device; a shoulder replacement device; a wrist replacement device; an ankle replacement device; and an inter-vertebral disk replacement device (artificial disk device).

In an embodiment, the methods disclosed herein include an induction regimen that is administered locally to a site of pain, to a site of inflammation, to an organ, to a joint, or to the spine.

In an embodiment, the methods disclosed herein include an induction regimen where the route of administration is selected from: intra-operative, intracerebral, intracerebroventricular, into an organ selected from intracardiac, intraventricular, and intracoronary administration; endoscopic retrograde cholangiopancreatography; intrapleural, intraperitoneal, intradiskal administration; intra-articular or intracapsular administration; peridiskal administration; pericapsular administration; intramedullary administration; intrathecal administration; epidural administration (including periradicular and transforaminal administration); intra-facet administration; intra-cartilaginous administration; and epidural, intrapleural, or intraperitoneal administration. In this embodiment, the maintenance regimen route of administration is selected from: IV, perispinal, intramuscular, SC, oral, intranasal, buccal; inhalation (including intrapulmonary and intrabronchial); and transdermal administration.

In an embodiment, the induction regimen is administered locally to a site in or adjacent to one or more intervertebral disks, in or adjacent to one or more vertebra(e), or adjacent to one or more spinal nerve root(s) or nerve(s); or is administered using intra-operative administration. This intra-operative administration includes administration into or adjacent to one or more spinal structure(s) selected from spinal NR(s) or nerve(s), intervertebral disk(s), vertebra(e), and dura.

In an embodiment, the maintenance regimen is administered using IV, perispinal, intramuscular, SC, oral, intranasal, buccal; inhalation (including intrapulmonary and intrabronchial); and transdermal administration. In one aspect, the maintenance regimen may be administered using a catheter and a pump, or by implantation of a depot formulation, controlled-release, or hydrogel formulation.

It may also be useful to administer the induction regimen using intradiskal, peridiskal, epidural (including periradicular and transforaminal), intradiskal/peridiskal, intradiskal/epidural, intradiskal/peridiskal/epidural or intra-facet administration, and the maintenance route of administration is selected from; IV, perispinal, intramuscular, SC, oral, intranasal, buccal; inhalation (including intrapulmonary and intrabronchial); and transdermal administration. It is also conceived that the maintenance regimen comprises implantation of a depot formulation, controlled-release, or hydrogel formulation. In other circumstances, it may be useful to administer the induction regimen via implantation of a depot formulation, controlled-release, or hydrogel formulation.

In another embodiment, the induction regimen is administered using epidural administration and the maintenance route of administration is selected from: IV, perispinal, intramuscular, SC, oral, intranasal, buccal, inhalation (including intrapulmonary and intrabronchial); and transdermal administration. In this scenario, the induction regimen may also involve implantation of a depot formulation, controlled-release, or hydrogel formulation. Likewise, the maintenance regimen comprises implantation of a depot formulation, controlled-release, or hydrogel formulation.

In an embodiment of the methods disclosed herein, the induction regimen is completed prior to beginning administration of the maintenance regimen. Alternatively, the maintenance regimen may begin at or near the same time as the induction regimen.

In an additional embodiment of the methods disclosed herein, the induction regimen direct TNF-I and the maintenance regimen direct TNF-I are the same.

In an alternative embodiment, the induction regimen may include an NFκB-I and the maintenance regimen may include a different NFκB-I.

In one embodiment, the direct TNF-I is selected from the group consisting of an antibody or antibody fragment, a fusion protein, a peptide, a SMIP, a small molecule, an oligonucleotide (such as an siRNA), an oligosaccharide, a soluble cytokine receptor or fragment thereof, a soluble TNF receptor Type I or a functional fragment thereof, a polypeptide that binds to TNF, and a dominant negative TNF molecule. In one aspect, the direct TNF-I is selected from the group consisting of is selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP).

In one embodiment, the NFκB-I is selected from the group consisting of sulfasalazine, sulindac, clonidine, helenalin, wedelolactone, pyrollidinedithiocarbamate (PDTC), IKK-2 inhibitors, and IKK inhibitors.

The methods disclosed herein also consider additionally administering to the subject a therapeutically effective amount of a supplemental active ingredient (SAI). This SAI may be selected from the group consisting of a second TAT, a corticosteroid, ozone, an antirheumatic drug, a local anesthetic, a neuroprotective agent, a salicylic acid acetate, a hydromorphone, an NSAID, a cox-2 inhibitor, an antidepressant, an anticonvulsant, a calcium channel blocker, and an antibiotic.

In an embodiment, herein disclosed is a kit. This kit includes but is not limited to one or more of the following: a) at least one container comprising an induction regimen of a direct TNF-I or an NFκB-I; b) a delivery vehicle to administer the induction regimen of a direct TNF-I or an NFκB-I; c) instructions for administration of the induction regimen of a direct TNF-I or an NFκB-I.

In one aspect, the delivery vehicle included in the kit is selected from the group consisting of a syringe, a needle, a catheter, or a pump. In another aspect, the kit may additionally comprisr at least one SAI.

In an embodiment, the delivery vehicle in the kit may be adapted for an induction regimen to be administered using intra-operative administration. Alternatively, the delivery vehicle included in the kit may be adapted for an induction regimen to be administered using an intradiskal, peridiskal, or epidural (including periradicular and transforaminal) administration, or any combination thereof, or intra-facet administration.

In an embodiment, herein disclosed is a kit, including the following: a) at least one container comprising an induction regimen of a direct TNF-I or an NFκB-I; b) a delivery vehicle to administer the induction regimen of a direct TNF-I or an NFκB-I; c) at least one container comprising a maintenance regimen of a direct TNF-I or an NFκB-I; d) a delivery vehicle to administer the maintenance regimen of a direct TNF-I or an NFκB-I; and e) instructions for administration of the induction regimen of a direct TNF-I or an NFκB-I and the maintenance regimen of a direct TNF-I or an NFκB-I.

Unless otherwise defined, all technical and scientific terms used herein have the meaning commonly understood by one of ordinary skill in the art to which this invention pertains. All publications, patent applications, patents, and other references mentioned herein are incorporated by reference in their entirety. In case of conflict, the present specification, including definitions, will control. The disclosed materials, methods, and examples are illustrative only and not intended to be limiting. Skilled artisans will appreciate that methods and materials similar or equivalent to those described herein can be used to practice the invention.

### BRIEF DESCRIPTIONS OF THE DRAWINGS

FIG. 1 demonstrates the ICs and IMs to which the TATs as described herein are directed.

FIG. 2 demonstrates the designated IC polypeptides TNF and IL-I and the defined polypeptides of the TNF and IL-1 pathways.

FIG. 3 sets forth representative TNF-I doses for induction and maintenance regimens in pain patients using Humira® (adalimumab) or Enbrel® (etanercept).

FIG. 4 sets forth representative TNF-I doses for induction and maintenance regimens in pain patients using Remicade® (infliximab).

FIG. 5 sets forth representative TNF-I doses for induction and maintenance regimens in pain patients using Cimzia® (certolizumab pegol, CDP870).

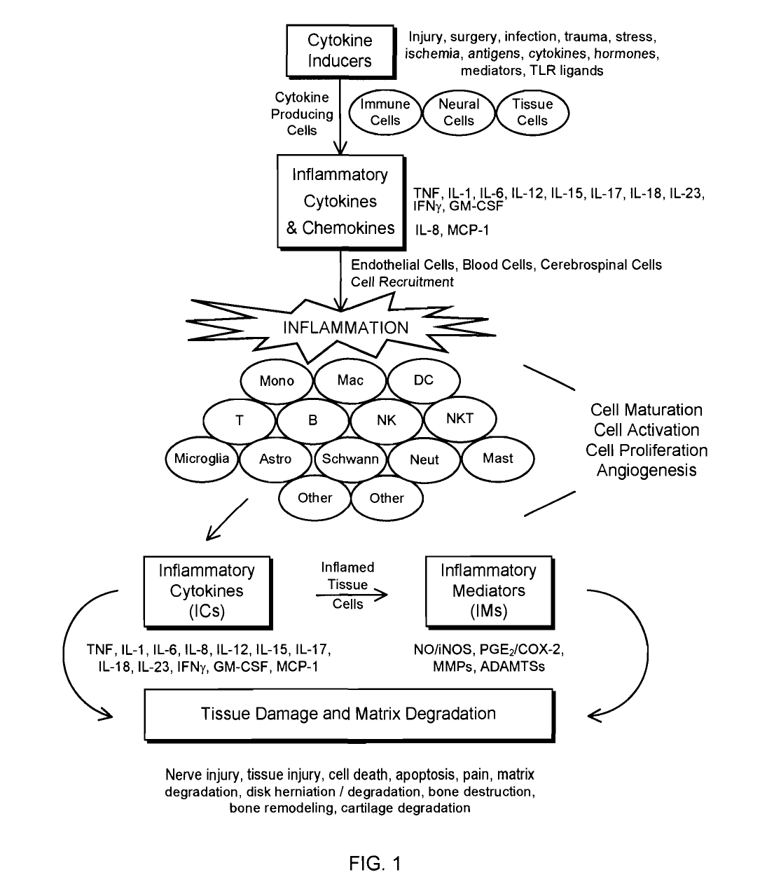

### DETAILED DESCRIPTION OF THE INVENTION

### I. Definitions

As used herein, the terms “tumor necrosis factor,” “tumor necrosis factor-alpha,” “TNF,” and “TNF-α” are used interchangeably to refer to a naturally occurring cytokine, which plays a key role in the inflammatory response, in the immune response and in the response to infection. The term “human TNF” (abbreviated as huTNF or hTNF), as used herein, is intended to refer to a human cytokine that exists as a 17 kiloDalton (kD) secreted form and a 26 kD membrane associated form, the biologically active forms of which are composed of trimers of noncovalently bound 17 kD or 26 kD molecules respectively.

As used herein, the term “inflammatory cytokine” is used interchangeably with “IC” and refers to one of the following designated polypeptides: TNF, IL-1, IL-6, IL-8, IL-12, IL-15, IL-17, IL-18, IL-23, IFN-γ, GM-CSF, MCP-1, IL-8 and MCP-1.

As used herein, the term “inflammatory mediator” is used interchangeably with “IM” and refers to one of the following: MMP-1 (collagenase-1), MMP-2 (Gelatinase A), MMP-3 (stromelysin), MMP-7 (Matrilysin), MMP-9 (gelatinase), MMP-13 (collagenase-3), ADAMTS4, ADAMTS5, iNOS, NO, COX-2, and PGE2.

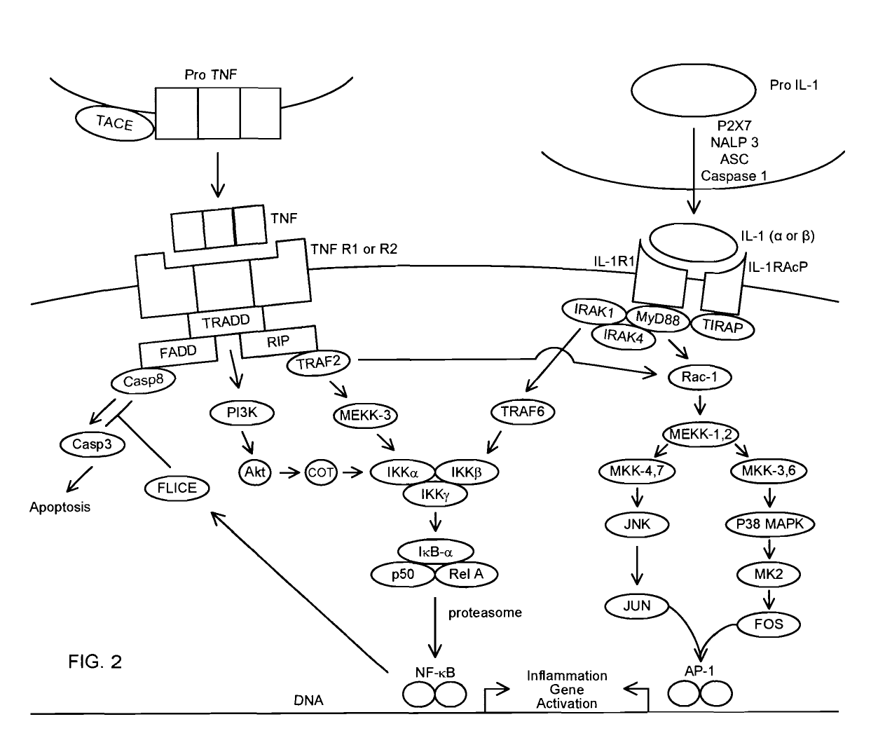

As used herein, the terms “inflammatory cytokine inhibitor” and “IC-I” are used interchangeably and refer to any molecule that blocks, suppresses or reduces gene expression, protein production and processing, protein release, and/or biological activity of: a) one of the following designated polypeptides: TNF, IL-1, IL-6, IL-12, IL-15, IL-17, IL-18, IL-23, IFNg, GM-CSF, and IL-8 (CXCR8) and MCP-1 (CCL2), or the designated polypeptide's biological receptor, coreceptor, or coligand, as described above, or b) one of the defined polypeptides within the designated polypeptide's pathway, as described above and described further below. See also, e.g., FIG. 2 for a depiction of the defined polypeptides in the TNF and IL-1 pathways.

An IC-I can be a “direct IC-I,” meaning a molecule (e.g., an antibody (Ab) or fusion polypeptide) that binds directly to and inhibits the biological activity of a designated polypeptide, its receptor, coreceptor, or coligand, or is a molecule (e.g., a nucleic acid such as an siRNA or antisense molecule) that binds directly to a nucleic acid molecule encoding the designated polypeptide or its receptor, coreceptor, or coligand and inhibits or reduces the expression of the designated polypeptide or its receptor, coreceptor, or coligand.

As used herein, the terms “inflammatory mediator inhibitor” and “IM-I” are used interchangeably and refer to any molecule that blocks, suppresses or reduces gene expression, protein production and processing, protein release, and/or biological activity of one of the following IMs: MMP-1 (collagenase-1), MMP-2 (Gelatinase A), MMP-3 (stromelysin), MMP-7 (Matrilysin), MMP-9 (gelatinase), MMP-13 (collagenase-3), ADAMTS4, ADAMTS5, iNOS, NO, COX-2, and PGE2. An IM-I can be a “direct IM-I,” meaning a molecule (e.g., an Ab, fusion polypeptide, or small molecule) that binds directly to and inhibits the biological activity of MMP-1 (collagenase-1), MMP-2 (Gelatinase A), MMP-3 (stromelysin), MMP-7 (Matrilysin), MMP-9 (gelatinase), MMP-13 (collagenase-3), ADAMTS4, ADAMTS5, iNOS, NO, COX-2, or PGE2, or meaning a molecule (e.g., a nucleic acid such as an siRNA or antisense molecule) that binds directly to a nucleic acid molecule encoding any of the foregoing IMs, inhibiting or reducing its expression.

Unless otherwise indicated, “small molecule,” and “small molecule inhibitor” are used interchangeably to refer to a molecule of low relative molecular mass that blocks, suppresses or reduces biological activity of a designated polypeptide. The term “low relative molecular mass” has art-recognized meaning, and refers to a molecule having a relative small number of atoms, typically less than 100 atoms (as compared to a protein, “biologic” or “macromolecule”). A small molecule can have a molecular weight of about 100 to 5000 daltons, e.g., about 500 to about 2000 daltons, or about 500 to about 1200 daltons.

As used herein, the terms “non-operative treatment” and “conventional non-invasive treatments” and “conservative care” are used interchangeably and mean one or more of watchful waiting by a healthcare provider, exercise, bed rest or reduced activity, physical therapy, administration of an NSAID, administration of a steroid, the use of an orthotic brace, and administration of oral analgesics including opioid analgesics.

As used herein, the term “peri-operative” means relating to, occurring in, or being the period around the time (e.g., before, during, and/or after) of a surgical operation.

“Interspinous route” refers to parenteral injection through the skin in the midline, in the interspace between two spinous processes, to deliver the therapeutic agent(s) in anatomic proximity to the spine.

“Intrathecal” means injection into the spinal canal (intrathecal space surrounding the spinal cord).

“Epidural” means in the space between the pia and dura mater, in which the nerve roots typically are found. “Periradicular” and “transforaminal” refer to specific types of epidural administration. “Periradicular” means within the epidural space, specifically in the region of the radicles (nerve roots). “Transforaminal” means through the vertebral foramen and within the epidural space, specifically in the region of the radicles. The terms “radicle” and “nerve root” are used interchangeably.

“Intradiskal” means penetration of the outer wall and into the nucleus pulposus of a disk and/or into the annulus fibrosus of a disk.

“Peridiskal” means adjacent to an outer wall of the annulus fibrosus; outside but closely adjacent to an outer wall of the annulus fibrosus; and/or outside but closely adjacent to an endplate of an adjacent vertebral body.

“Perispinal” means in the paraspinal muscles near the spine.

“Intradiskal/epidural” means a combination of intradiskal, as defined above, and epidural, as defined above. For example, an “intradiskal/epidural” administration of a TAT could include administration of the TAT into the nucleus pulposus of a disk and administration of the TAT into the epidural space, e.g., using a needle adapted for intradiskal administration to administer the TAT intradiskally, followed by injection epidurally, either with the same or a different needle.

“Intradiskal/peridiskal” means a combination of intradiskal, as defined above, and peridiskal, as defined above. For example, an “intradiskal/peridiskal” administration of a TAT could include administration of the TAT into the nucleus pulposus of a disk and administration of the TAT into the peridiskal space adjacent to an outer wall of the annulus fibrosus, e.g., using a needle adapted for intradiskal administration to administer the TAT intradiskally, followed by injection peridiskally, either with the same or a different needle.

“Intradiskal/peridiskal/epidural” means a combination of intradiskal, peridiskal, and epidural, as defined above. For example, an “intradiskal/peridiskal/epidural” administration of a TAT could include administration of the TAT into the nucleus pulposus of a disk and administration of the TAT into the peridiskal space adjacent to an outer wall of the annulus fibrosus, and further administration of a TAT into the epidural space.

“Intracerebroventricular” means into one of the cerebral ventricles.

“Intracerebral” means within the cerebrum.

“Intracardiac” means within the heart.

“Intraventricular” means within a ventricle.

“Intracoronary” means within the coronary arteries.

“Intra-articular” means in the articular space within the joint capsule.

“Intracapsular” (sometimes referred to as intra-articular) means inside the joint capsule, including but not limited to the intra-articular space.

“Pericapsular” means outside but closely adjacent to the outer wall of the capsule.

“Intramedullary” means within the marrow cavity of a bone.

“Intra-cartilaginous” means within a cartilage; endochondral.

“Intraileal” means within the distal portion of the small intestine, from the jejunum to the cecum.

“Endoscopic Retrograde Cholangiopancreatography (ERCP)” means a procedure that is used to diagnose problems in the liver, gallbladder, pancreas and bile ducts that combines the use of x rays and an endoscope, which is a long, flexible, lighted tube with a distal camera. Through the endoscope, the physician can visualize the inside of the stomach and intestine, and inject dyes into the biliary and pancreatic ducts so that disorders of these organs can be seen radiographically.

“Intravenous regional perfusion” means a procedure used to treat Complex Regional Pain Syndrome in which a therapeutic agent is infused intravenously into a limb made ischemic through the use of an arterial tourniquet. The therapeutic agent is left in contact with the affected limb for a period of time of up to 30 minutes, following which perfusion is restored to the limb by slowly letting down and then removing the tourniquet, and the high concentrations of the therapeutic agent are allowed to mix with venous blood from the rest of the body.

“Intrapulmonary” means within the lungs or the lungs' bronchi.

“Intrabronchial” means within a bronchus.

As used herein, an “induction regimen” has the following properties: it is administered by: 1) a more invasive route of administration than a maintenance regimen or more local site of administration than a maintenance regimen; and 2) a lower dose per administration than the dose per administration used in the maintenance regimen administered to the same subject, concurrent with or following the induction regimen.

As used herein, “treatment” means any manner in which one or more of the symptoms of a disease or disorder are ameliorated or otherwise beneficially altered. As used herein, amelioration of the symptoms of a particular disorder refers to any lessening, whether permanent or temporary, lasting or transient that can be attributed to or associated with treatment by the methods of the present invention.

A “therapeutically effective amount” is an amount sufficient to affect a beneficial or desired clinical result, such as prevention or treatment of injury and/or pain; the prevention, delaying, postponement, reduction, or elimination of the need for an invasive surgical procedure; or an improvement in the outcome of a subject that undergoes an invasive procedure.

As used herein, “delaying” or “postponing” are used interchangeably and mean to defer, hinder, slow, retard, and/or stabilize a subject's need for or eligibility for an invasive surgical procedure. This delay can be of varying lengths of time, depending on the history of the disease and/or individuals being treated. As is evident to one skilled in the art, a sufficient or significant delay can, in effect, encompass prevention, in that the individual does not need the procedure. A method that “delays” or “postpones” exhibition of the need for or the eligibility for the invasive procedure is a method that reduces probability of the need for or the eligibility for the procedure in a given time frame, when compared to not using the method. Such comparisons can be based on clinical studies, using a group of subjects sharing similar disease characteristics.

As used herein, a method for “improving the outcome” of an invasive procedure refers to a method that, for example, reduces severity or intensity of pain, symptoms, or disability, results in alleviation of one or more symptoms associated with the disease or disorder, reduces resting pain and/or mechanically-induced pain, shortens the duration of pain, symptoms, or disability, and/or reduces pain sensitivity or sensation, in a given time frame after the procedure when compared to the outcome observed when not using the recited method. Other examples of improved outcome are set forth further herein. Such comparisons can be based on clinical studies, using a group of subjects sharing similar disease characteristics.

As used herein, and unless otherwise indicated, the terms “patient,” “subject,” and “individual” are used interchangeably to refer to a vertebrate, and particularly a mammal including, without limitation, humans, farm animals, sport animals, pets, primates, horses, dogs, cats, mice and rats.

As used herein, the term “invasive,” when in the context of administration of a TAT, refers to the degree to which a particular administration regimen or mode of administration involves penetration of the delivery vehicle into the body, organ, or internal structures. A more invasive mode of administration refers to greater penetration into the body, organ, or internal structures than a less invasive mode. For example, a more invasive mode of administration can be evidenced through use of a longer needle, e.g., to penetrate further into the body, organ, or internal structures. Thus, intramuscular administration is more invasive than subcutaneous (SC) as the administration is deeper into the body. A more invasive mode of administration can be evidenced by the use of a catheter to administer into an internal organ, artery, or vein. A more invasive mode of administration can be evidenced by the requirement for local anesthesia during the procedure, e.g., to minimize accompanying pain directly due to the invasive procedure. A more invasive mode can be evidenced by a requirement for image guidance (e.g., ultrasound or radiographic imagery to guide the procedure) for the procedure (e.g., flouroscopy for epidural or intradiskal administration). In some cases, a more invasive mode can involve greater risk, discomfort, or inconvenience to subject.

The following modes of administration are listed in order of invasiveness from highest to lowest: intra-operative, meaning into a surgical wound, to directly influence inflammation at the site of the surgical wound or organ manipulation (e.g. into the wound in the region of the NR or disk during a spine surgical procedure; into the wound in the region of the ileus or other bowel segment, or other abdominal organ during an abdominal surgery, and so forth); intracerebral and intracerebroventricular; into an organ such as the heart, kidney, liver, pancreas and so forth, e.g., during a percutaneous intracardiac administration (e.g., intraventricular and intracoronary administration); endoscopic retrograde cholangiopancreatography (ERCP); intradiskal; peridiskal, and intrathecal; epidural, including periradicular and transforaminal; intramedullary; intra-cartilaginous, intraarticular and intracapsular, and intra-facet; pericapsular; intravenous (IV); perispinal, intramuscular; SC; and all other non-invasive modes of administration, including oral, intranasal, buccal, via inhalation/aerosol (including intrapulmonary and intrabronchial), and transdermal.

The term “pain” includes nociception and the sensation of pain, both of which can be assessed objectively and subjectively, using pain scores and other methods well-known in the art. Pain, as used herein, includes allodynia (i.e., increased response to a normally non-noxious stimulus) and hyperalgesia (i.e., increased response to a normally noxious or unpleasant stimulus), which can in turn, be thermal or mechanical (tactile) in nature. In some embodiments, pain is characterized by thermal sensitivity, mechanical sensitivity and/or resting pain. In other embodiments, pain comprises mechanically-induced pain or resting pain. In still other embodiments, the pain comprises resting pain. The pain can be primary or secondary pain, as is well-known in the art. Exemplary types of pain preventable or treatable by the methods of the present invention include, without limitation, back pain in the lumbar regions (low back pain) or cervical region (neck pain), leg pain, arm pain, radiating low back (“sciatic pain”), radiating or “radicular” pain in the distribution of an affected NR (experienced in the lower back and leg from lumber pathology, or in the neck and arm from cervical pathology), and neuropathic pain of the arm, neck, back, lower back, leg, and related pain distributions resulting from disk and spine pathology.

As used herein, “neuropathic pain” means pain arising from injury to the NR, dorsal root ganglion or peripheral nerve. Such pain can be caused by neuronal injury arising from e.g., compression, trauma, viral, toxic, or metabolic insults affecting sensory nerve fibers or neurons. Examples of compression include compression by tumor, disk, carpal tunnel, Saturday night palsies, and other. Traumatic injuries include accidents such as automobile, airplane, and other vehicle accidents or sports injuries such as diving and horse-back riding. Viral insults include herpetic infections causing acute zoster or “shingles”, and post-herpetic neuralgia. Toxic injuries include injuries caused by diverse chemotherapeutic agents. Metabolic injuries include diabetic neuropathy. Resulting neuropathic pain conditions include but are not limited to sciatica, radiating low back pain, radiating neck and arm pain, post-herpetic neuralgia, pain of chemotherapy induced neuropathy, and other similar types of persistent pain.

As used herein, “post-surgical pain” and “surgery-induced pain” are used interchangeably, and refer to pain arising in the recovery period of days or weeks following a surgical procedure. Specific examples of such pain that occur with increased frequency after spinal surgery include, without limitation, leg pain, back pain, neck pain, and/or arm pain. Specific examples of such pain that occur with increased frequency after, for example, spinal surgery include, without limitation, leg pain, back pain, neck pain, and/or arm pain. “Resting pain” refers to pain occurring even while the individual is at rest as opposed to, for example, pain occurring when the individual moves or is subjected to other mechanical stimuli. “Mechanically-induced pain” (interchangeably termed mechanosensory pain) refers to pain induced by a mechanical stimulus, such as the application of weight to a surface, tactile stimulus, and stimulation caused or associated with movement (including coughing, shifting of weight, etc.).

### II. Novel Induction and Maintenance Regimens

The present methods include the use of an induction and a maintenance regimen for administration of a TAT. For example, the methods comprise administering to the subject an induction regimen of a therapeutically effective amount of a TAT (e.g., a TNF-I); and administering to the subject a maintenance regimen of a therapeutically effective amount of a TAT, e.g., a TNF-I. An induction regimen and a maintenance regimen can independently include multiple administrations of a TAT (e.g., 2, 3, 4, 5, 6, 8, 10, or more separate administrations). In some embodiments, a maintenance regimen will comprise more separate administrations of a TAT than an induction regimen. For example, an induction regimen may comprise one administration of a TAT (e.g., a single intracerebroventricular administration), while a maintenance regimen may comprise weekly or monthly intramuscular injections for a period of 6 months to a year, or longer.

An induction regimen can involve a more invasive route of administration than a maintenance regimen. A more invasive route of administration can be evaluated according to the invasiveness spectrum defined previously. Thus, an induction regimen, in some cases, can include a mode of administration selected from, for example, intracerebral, intracerebroventricular, intracardiac, intraileal, intrathecal, intradiskal, intracapsular, intra-articular or intra-facet, epidural (including transforaminal and periradicular), or perispinal, while a maintenance regimen can be selected from, for example, perispinal (provided the induction regimen is not perispinal), intra-cartilaginous, IV, intramuscular, or SC administration.

An induction regimen can involve a more local or targeted administration than a maintenance regimen. A more local administration can be obtained by targeting the administration to the site of pain, inflammation, or injury, or in close proximity to the site of pain, inflammation, or injury in the subject. Local administration can be within 10 cm of the site of injury, e.g., within 10, 9, 8, 7, 6, 5, 4, 3, 2, 1, or 0.5 cm. Modes of administration that result in “systemic” or “parenteral” (systemic non-oral) administration are understood by those having ordinary skill in the art to be “non-local” and non-targeted. Thus, in some cases, an induction regimen will include administration in proximity to the site of pain, inflammation, or injury, while the maintenance regimen will involve systemic administration. For example, an induction regimen can involve epidural administration, while a maintenance regimen can involve systemic administration, e.g., through IV, intramuscular, or SC administration.

An induction regimen comprises a lower dose per administration of a TAT than a maintenance regimen. The dose per administration can be evaluated by those having ordinary skill in the art. Typically, the lower dose per administration of an induction regimen is less than about 50% of the maintenance dose per administration, e.g., less than about 50%, 45%, 40%, 35%, 30%, 25%, 20%, 15%, 10%, or 5% of the maintenance dose per administration.

In particular embodiments, an induction regimen may comprise local, invasive administration (including intra-articular, intracapsular, pericapsular, or intra-facet) of one or more low doses per administration (low as compared to the dose per administration of the maintenance regimen) of at least one TAT, e.g., in an amount sufficient to provide clinically meaningful relief of pain or other symptoms. In preferred embodiments, an “induction regimen” comprises one to seven administrations (e.g., 1, 2, 3, 4, 5, 6 or 7) of at least one TNF inhibitor (TNF-I) selected from the group consisting of Enbrel® (etanercept); Humira® (adalimumab); Cimzia® (certolizumab pegol); Remicade® (infliximab) or an NFκB inhibitor (NFκB-I) selected from the group consisting of sulfasalazine, sulindac, clonidine, helenalin, wedelolactone, pyrollidinedithiocarbamate (PDTC), and others, e.g., those set forth in US Pat. Publication 2006/0253100.

In preferred embodiments, the more local and/or more invasive route of administration of an induction regimen results in a higher concentration of drug in or at the presumed site of therapeutic action or pathology, such as the affected nerve root.

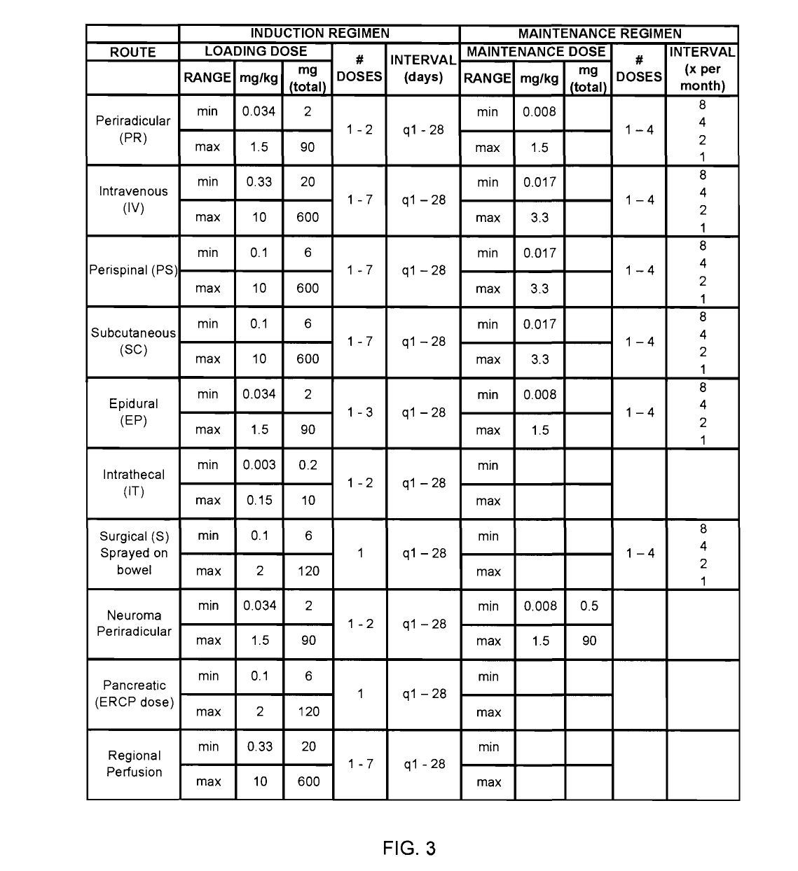

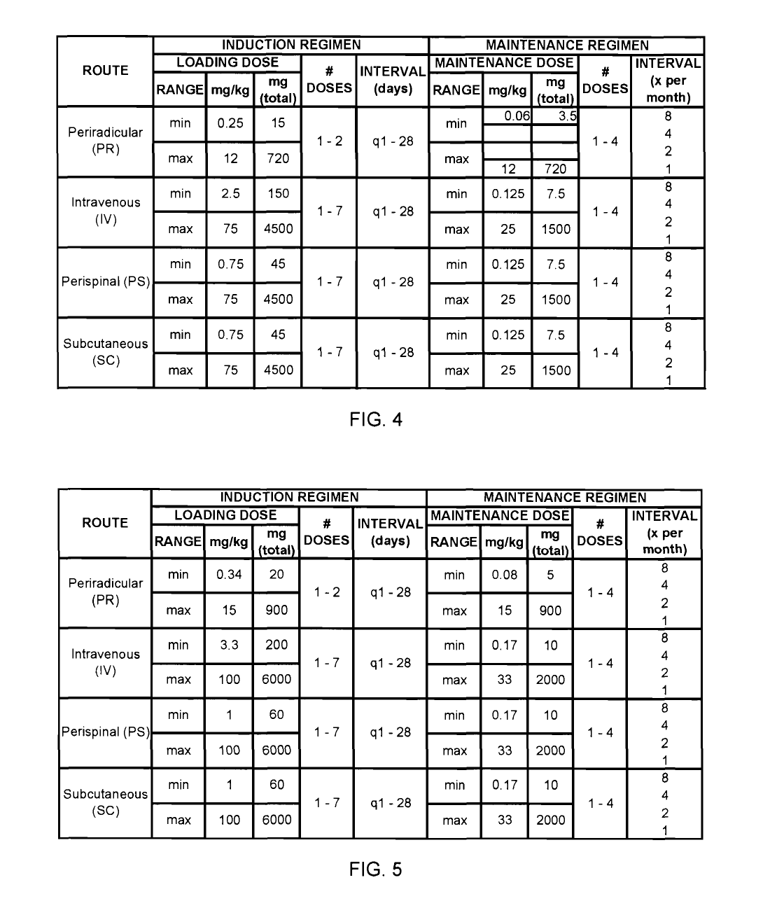

Preferred dosage ranges for an “induction regimen” of a TAT will vary depending upon clinical factors observed by the clinician, the indication, and the particular TAT, and will generally comprise administration of a “loading dose” of at least one TAT, or a dose which will generally achieve clinically meaningful induction of tissue protection or relief of pain upon administration. In preferred embodiments, the induction regimen will provide protection from injury or relief of pain or other symptoms within several hours of administration. In some embodiments, the induction regimen comprises administration of a “loading dose” of at least one TAT (e.g., TNF-I) via local administration, for example via intracerebral, intracerebroventricular, intraventricular, intracoronary, endoscopic retrograde cholangiopancreatography (ERCP), intradiskal, intracapsular, intra-articular, peridiskal, pericapsular, intra-facet, intramedullary, intrathecal, epidural, periradicular, transforaminal, intra-cartilaginous, intravenous (IV), perispinal, intramuscular, and IV regional perfusion administration. Preferred induction regimens for several currently marketed TNF-Is are provided in FIGS. 3-5.

A “maintenance regimen” of a TAT will also vary depending upon clinical factors observed by the clinician, the indication, and the type of inhibitor, and will generally comprise administration of a “maintenance dose” of at least one TAT (e.g., TNF-I), or a dose which will generally achieve durable induction of protection from neuronal insult or relief from pain when administered concurrently with and/or subsequent to, administration of an “induction regimen.” A “maintenance regimen” of a TAT may be administered once, or may be administered periodically (e.g., daily, weekly, monthly, bimonthly) according to a dosage regimen prescribed by the treating physician. In some embodiments, the maintenance regimen comprises administration of a maintenance dose of at least one TAT via a less invasive or less local mode of administration that an induction regimen but that is still effective for durable induction of protection from neuronal insult or relief from pain. For example, a maintenance dose of TATs will preferably be administered via less invasive modes of administration, such as IV, intramuscular, or SC administration. In some embodiments, the maintenance regimen comprises administration of at least one maintenance dose via continuous dosage means, such as a pump and catheter. The catheter may be inserted during the course of administering the induction regimen, or may be separately inserted. Preferred maintenance regimens for several approved TNF-Is are provided in FIGS. 3-5.

Routes of administration, timing of administration, and choice of TAT for the “induction regimen” and “maintenance regimen” will vary depending upon the practitioner's choice of regimen, the indication, and the type of inhibitor. The criteria that might lead a skilled practitioner to choose a particular TAT for a particular regimen will often include drug concentration, lipophilicity, solubility, half life, formulation characteristics, pH, pKa, known adverse events profile, tmax, potency, and affinity (e.g., for the target), among other factors. The relative weight and strength of the applicability of each of these criteria would depend, in part, on the indication and on the site of administration. Thus, for example, since a limited volume of agent can be safely injected intradiskally, an agent high in concentration might be chosen to maximize the dosage given. In an epidural route of administration, a lipophilic agent might limit spread of the TAT to distant, non-pathologic locations within the epidural space, while choice of a large protein TAT or a depot formulation might limit migration out of the epidural space. Moreover, in certain embodiments, the induction regimen is administered and completed prior to beginning administration of the maintenance regimen. In others, the maintenance regimen may begin at or near the same time as the induction regimen.

In cases where a surgical intervention is associated with the treating of a disease or disorder, the induction and/or maintenance regimen can also involve optional temporary peri-operative interruption of the treatment course with the TAT, e.g., TNF-I, e.g., for a time period prior to and/or after the surgical intervention procedure. Such optional interruption is provided in order to address any perceived risk of increased infection risk upon administration of a TAT peri-operatively, with resumption of the TAT treatment regimen post-operatively. Peri-operative interruption of therapy would be at the discretion of the clinician responsible for managing the patient's therapy before, during, and/or after the invasive spinal procedure. The optional interruption time period prior to and/or after the invasive spinal procedure can be about equivalent or can be different. An optional interruption time period can range from about 1 day to about 14 days, or any time there between (e.g., 2, 4, 6, 8, 10, 12 days). In some embodiments, the optional interruption time period prior to and/or after the invasive spinal procedure is equivalent to about 1 to about 4 half-lives (t_{1/2}) (e.g., 1, 2, 3, or 4 half-lives) of the TAT in serum. Typically, the optional interruption period will be longer prior to the invasive procedure than after the invasive procedure.

The TAT for use in the maintenance regimen may be the same as or different than the TAT for use in the induction regimen. The formulation of the TATs can be the same or different, e.g., both can be an aqueous formulation, or one could be aqueous while the other is an oil-in-water emulsion, or one could be aqueous while the other could be a depot or controlled-release formulation.

In an embodiment, the induction regimen and/or maintenance regimen may be administered by means of a catheter and pump system, such as a fully implantable pump system or an external pump system. Suitable pump and catheter systems are commercially available, e.g., SynchroMed® pump and InDura® intrathecal catheters (both from Medtronic Sofamor Danek, Memphis, Tenn.). The induction and/or maintenance regimen may also be administered as part of an implantable device that comprises a depot formulation of one or more TATs. In some embodiments, the device comprising a depot formulation may take the form of a biodegradable or resorbable substance, including polymers such as poly lactic acid, (PLA), polyglycolic acid (PGA), a hydrogel, and co-polymers of polylactic acid/polyglycolic acid (PLGA). The device comprising a depot formulation may comprise capsules or microcapsules. In a further embodiment, the maintenance regimen may be administered by transfusion, such as IV transfusion.

The maintenance dose per administration, as practiced in the invention, is higher than the more invasive and/or localized induction dose per administration in the induction regimen. However, the maintenance dose is still lower than would be required for an induction regimen performed without the invasive induction phase as practiced in the invention. Thus, the invention offers an improvement in treatment of spinal disorders, over a regimen in which the induction phase simply uses an systemic dose that is higher per administration, or more frequent, followed by maintenance with a lower systemic dose.

### III. Conditions Treatable Using the Novel Regimens

TNF and other ICs and IMs are closely associated with the initiation or exacerbation of various disorders, such as pain, including neuropathic, radicular, nociceptic pain, complex regional pain syndromes (CRPS) Types I and II; spinal disorders; and inflammatory conditions and chronic inflammatory conditions, including chronic obstructive airway disease, such as asthma and chronic obstructive pulmonary disease (COPD). For a description of TNF mediated disorders, see Tobinick et al., US Patent Publication 2003/0009772 and Banerjee et al., US Patent Publication 2004/0126372, the disclosures of which are hereby incorporated herein by reference.

In an embodiment, the present invention provides novel therapeutic regimens for treating a disease or disorder in which IC or IM activity is implicated as causing, contributing to, exacerbating, or perpetuating the pathophysiology of the disease or disorder.

A. Pain

The present invention provides novel regimens for the treatment of any type of pain syndrome. Such pain syndromes include, for example, acute and chronic pain, such as complex regional pain syndrome (CRPS) type I and type II, neuropathic pain, post-operative pain, pain caused by inflammation, chronic lower back pain, cluster headaches, herpes neuralgia, phantom limb pain, central pain, dental pain, opioid-resistant pain, visceral pain, surgical pain, bone injury pain, pain during labor and delivery, pain resulting from burns (e.g., sunburn), post partum pain, migraine, angina pain, fibromyalgia, and genitourinary tract-related pain, including cystitis. As described above, the term “pain” as used herein refers to all types of pain or nociception.

B. Spinal Disorders

The present invention provides novel regimens for the prevention or treatment of any type of spinal disorder, including, for example, inter-vertebral disk herniation (HD), spinal stenosis (SS), spinal instability, degenerative disk disease (DDD), either with or without internal disk derangement and/or diskogenic pain, facet joint disease, nerve entrapment, FBSS, radicular pain or radiating pain, sciatica, arthritic spinal disorders, nerve compression disorders, back or neck pain, low back pain, and chronic back pain, and spinal cord injury; see, e.g., co-pending U.S. applications Ser. No. ______ (Attorney Docket No. 21782-005001), and Ser. No. ______ (Attorney Docket No. 21782-006001), filed concurrently herewith. Many variations exist in the terms used for spinal disorders, and it will be obvious to one skilled in the art that closely related disorders by different terminology can be treated using the methods described herein. The intention is that the invention applies broadly to spinal disorders involving pain or degeneration of the disk, vertebrae and nervous system structures in and emerging from the spine.

Arthritic disorders that may be treated using the present invention include, for example, osteoarthritis, degenerative spinal arthritis, cervical arthritis, thoracic arthritis, DDD, facet disease, facet joint degeneration, facet joint pain, facet joint syndrome, and impinging facet joints.

Nerve compression disorders that may be treated using the present invention include, for example, bone spurs, osteophytes, nerve compression syndrome, lumbago, nerve root compression, neural foraminal narrowing, pinched nerve, and sciatica.

Spondylolisthesis and spondylosis disorders may be treated using the present invention include, for example, degenerative spondylolisthesis, lumbar spondylolisthesis, isthmic spondylolisthesis, grade 1 spondylolisthesis, spinal instability symptoms, slipped disk symptoms, ankylosing spondylosis, and degenerative spondylosis.

Other relevant spinal disorders that may be treated using the present invention include, for example, whiplash, whiplash associated disorders (WAD), whiplash headaches, cervical headaches, scoliosis, and scoliosis pain.

C. Orthopedic Disorders

The present invention provides novel regimens for the treatment of any type of orthopedic disorder. Orthopedic disorders include any acute, chronic, traumatic, and overuse injury or disorder of the musculoskeletal system. Orthopedic disorders that may be treated using the present invention include orthopedic joint disorders including hip, knee, shoulder, ankle, elbow, wrist, toe, finger, sacro-iliac, and spinal facet joint disorders.

In other embodiments, the present invention provides novel regimens for the treatment of patients to prevent, delay, postpone, reduce, eliminate, or improve the outcome of surgery, e.g., orthopedic surgery, such as knee arthroscopy and meniscectomy, shoulder arthroscopy and decompression, carpal tunnel release, knee arthroscopy and chondroplasty, removal of support implant, knee arthroscopy and anterior cruciate ligament reconstruction, knee replacement, knee arthroscopy repair of both menisci, hip replacement, shoulder arthroscopy/distal clavicle excision, repair of rotator cuff tendon, fracture repair (including femoral neck fracture, femoral shaft fracture, trochanteric fracture, ankle fracture (e.g., bimalleolar type and fibula type), bone/ulna fracture, and distal part of radius fracture), bone grafting, hand surgery, and sports' medicine surgeries. Additionally, the present invention may be used to improve the outcome of surgery, including a spinal decompression surgery (e.g., laminectomy or diskectomy) or a surgery to implant a device, including a stent, a pump, an orthopedic device, such as a spinal device (e.g., an annular repair device, a nucleus replacement device, a dynamic stabilization device, a synthetic bone graft substitute, an allograft cage, a motion preservation device, a pedicle screw, a facet screw, and a vertebral body replacement), a hip replacement device, a knee replacement device, and a shoulder replacement device. In a further embodiment, the present invention may be used to improve the outcome of surgery wherein one or more of the aforementioned devices is implanted in combination with, for example, a bone growth stimulation factor, a bioadhesive, an anti-adhesive device, a collagen sponge (e.g., a collagen sponge impregnated with a bioactive agent), a hydrogel, a gel, a resorbable material, ceramic granules, a bioactive agent, and combinations thereof.

The present invention provides novel regimens for the treatment of patients in which surgery has been performed but failed to achieve the desired clinical improvement. Disorders in which surgery may have failed to achieve the desired clinical improvement that may be treated using the present invention include, for example, failed back surgery syndrom, post-laminectomy syndrome, nerve entrapment, and implantation of an orthopedic device.

The present invention also provides novel regimens for the treatment of patients that have been deemed as eligible for hip and joint replacement surgery by a health care professional, or in which surgery has been performed.

D. Inflammatory Disease

The present invention provides novel regimens for the treatment of any type of inflammatory disorder or inflammatory disease. Inflammatory disorders are disorders in which an excessive or unregulated inflammatory response leads to excessive inflammatory symptoms, including signs of pain, heat, redness, swelling, host tissue damage, and loss of tissue function. The present invention is of particular use in patients with signs and symptoms of inflammatory disease associated with a specific condition.

Inflammatory disorders may be idiopathic and may or may not be immune mediated. Inflammatory disorders may be acute or chronic. Chronic inflammatory disorders are disorders in which an excessive or unregulated inflammatory response prolongs for weeks, months, years, or indefinitely. Chronic inflammatory disorders include chronic inflammatory disorders of the airways, bowel, connective tissues, joints, and skin.

Chronic inflammatory airway disorders that may be treated using the present invention include, for example, asthma (e.g., allergic asthma, non-allergic/intrinsic asthma, exercise-induced asthma, nocturnal asthma, occupational asthma, and steroid resistant asthma), exercise-induced bronchospasm (EIB), and chronic obstructive pulmonary disease (COPD).

Chronic inflammatory bowel diseases (IBD) that may be treated using the present invention include, for example, ulcerative colitis, and Crohn's disease.

Chronic inflammatory connective tissue diseases that may be treated using the present invention include, for example, lupus erythematosus, scleroderma, Sjogren's syndrome, poly- and dermatomyositis, vasculitis, and MCTD.

Chronic inflammatory joint diseases that may be treated using the present invention include, for example, rheumatoid arthritis (e.g., polyarthritis), juvenile chronic arthritis (Still's disease), rheumatoid spondylitis, lupus erythematosus, ankylosing spondylitis, psoriatic arthritis, and reactive arthritis. One aspect of the present invention provides novel regimens for treating chronic inflammatory joint diseases of the hip, including rheumatoid arthritis of the hip, bursitis of the hip, and osteoarthritis of the hip.

Chronic inflammatory skin diseases that may be treated using the present invention include, for example, psoriasis, diskoid lupus erythematosus, scleroderma, hives, rosacea, dermatitis, and atopic dermatitis (eczema).

The present invention is of particular use in patients with other diseases associated with inflammation, including, for example, spondyloarthropies, cardiomyopathy, atherosclerosis vasculitis (e.g., anti-neutrophil cytoplasmic Ab (ANCA)-associated vasculitis including chronic and relapsing ANCA-associated vasculitis), acute renal disease, chronic renal disease, glomerulonephritis, inflammatory eye disorders (e.g., retinitis), tuberculosis, chronic cholecystitis, bronchiectasis, Hashimoto's thyroidiitis, Silicosis and other pneumoconioses, and hyper-IgG4 disease.

The present invention also provides novel regimens for the treatment of ileus, including, for example, ileus induced by a surgical procedure.

The present invention is also useful in the treatment of inflammatory side effects associated with a pharmaceutical agent, wherein the inflammation is not associated with the pharmaceutical agent's desired effect and wherein TNF activity may be detrimental.

The present invention also provides novel regimens for the treatment of all patients undergoing surgery, in whom inflammation is known to contribute to deleterious post-operative conditions, including pain, healing time, recovery of bowel activity and function, and healing.

E. Immune System Disease

The present invention is of particular use in patients with immunologic disease, including clinical problems associated with an inappropriate immune response. Immunologic diseases that may be treated using the present invention include autoimmunity, transplant rejection, graft rejection, graft-versus-host disease, and hypersensitivity.

Autoimmunity is a tissue damaging immune response directed specifically and inappropriately against one or more self antigens. Autoimmune diseases that may benefit from treatment using the present invention include, for example, acute renal disease, chronic renal disease, pemphigus vulgaris, acute anti-neutrophil cytoplasmic Ab (ANCA)-associated vasculitis, acute disseminated encephalomyelitis (ADEM), Addison's disease, ankylosing spondylitis, antiphospholipid Ab syndrome (APS), aplastic anemia, autoimmune hepatitis, autoimmune oophoritis, coeliac disease, Crohn's disease, diabetes mellitus type I, goodpastures's syndrome, Grave's disease, Lupus erythematosus (e.g., systemic lupus erythematosus (SLE), lupus nephritis, and lupus cerebritis), and multiple sclerosis.

Hypersensitivity reactions or allergies are exaggerated, inappropriate, or prolonged immune responses that cause damage to otherwise normal tissue. Allergies that may benefit from treatment with the present invention include, for example, allergic rhinitis (hay fever), atopic dermatitis (eczema), allergic conjunctivitis, eosinophilic granuloma, septic shock, adult respiratory distress syndrome (ADSS), endotoxic shock, and respiratory distress syndrome.

F. Metabolic Disorders

The present invention provides novel regimens for the treatment of insulitis, pre-diabetes, diabetes, obesity, and diseases or disorders associated with diabetes and obesity.

Insulitis, including peri-insulitis and intra-insulitis, involves macrophage, dendritic cell, and B and T lymphocyte mediated destruction of insulin producing pancreatic B cells. It is considered a histopathological hallmark of type I (insulin-dependent) diabetes and typically progresses to overt diabetes.

Pre-diabetes, although not a type of diabetes, is a term used to refer to an intermediate metabolic stage between normal glucose homeostasis and diabetes. Pre-diabetes is a risk factor for future diabetes, i.e., within 10 years, and cardiovascular disease. Pre-diabetes may be diagnosed using, for example, a fasting plasma glucose test, an oral glucose tolerance test, and a random plasma glucose test.

Diabetes is a chronic, progressive, and systemic disease characterized by dysfunction in the metabolism of fats, carbohydrates, protein, and insulin, as well as dysfunction in the function and structure of blood vessels and nerves. Diabetes is frequently associated with diabetic neuropathy, peripheral neuropathy, diabetic retinopathy, diabetic ulcerations (e.g., skin ulcers), retinopathy ulcerations, diabetic macrovasculopathy, and poor wound healing.

In an embodiment, the present invention may be used to treat insulitis and pre-diabetes to prevent a subject's progression to diabetes. The present invention may also be particularly useful in the treatment of those conditions associated with diabetes, including diabetic neuropathy, peripheral neuropathy, diabetic retinopathy, diabetic ulcerations (e.g., skin ulcers), retinopathy ulcerations, diabetic macrovasculopathy. The present invention may be particularly useful for the treatment of skin ulcers and poor wound healing, including, chronic non-healing wounds.

Obesity, as used herein, refers to condition in which the subject has an excess of body fat relative to lean body mass. Obesity is frequently defined as a measure of an individual's body mass index (BMI). Normal individuals have a BMI range of 18.5 to 24.9; overweight 25.0 to 29.9; class I obesity 30 to 34.9; class II obesity 35.0 to 39.9; and class III obesity greater than 40. Obesity is frequently associated with various medical complications, including cancer of the cervix, colon, endometrium, gallbladder, prostate, and uterus; cardiovascular disease; diabetes; fatty liver; kidney disorders; gallbladder disease; dyslipidemia; respiratory tract infections; and gout.

In an embodiment, the present invention may be used to treat obesity and the conditions associated with obesity.

G. Cardiovascular Disease and Diseases of Endothelial Dysfunction

The present invention provides novel regimens for the treatment of any type of cardiovascular disease or disease of endothelial dysfunction. Cardiovascular diseases include, for example, coronary artery disease, angina pectoris, cardiomyopathy, atherosclerosis, myocardial infarction, cardiovascular tissue damage caused by cardiac bypass, cardiovascular damage caused by cardiac arrest, cardiogenic shock, atherosclerosis, restenosis, stenosis, coronary artery disease, valvular disease, congestive heart failure, reperfusion injury of the myocardium, and reperfusion injury of the brain. Cardiovascular disease may also include, for example, vasculitides (e.g., large vessel vasculitis, Takayasu's arteries, kawasaki's disease, and Behcet's disease). Diseases of endothelial dysfunction include not only the above but, for example, chronic kidney diseases, erectile dysfunction, polycystic ovarian disease, hypertension, and is associated with the development of rheumatoid arthritis and diabetes.

H. Disorders of the Central and Peripheral Nervous Systems

The present invention provides novel regimens for the treatment of any type of central nervous system (CNS) or peripheral nervous system disease. Diseases of the CNS include, for example, brain diseases (e.g., Alzheimer's disease, multiple sclerosis, Parkinson's disease, brain edema, inflammatory brain injury), spinal cord diseases, and Guillian Barre. Diseases of the peripheral nervous system include, for example, neuropathy (e.g., neuropathic pain, compressive neuropathy, ischemic neuropathy, diabetic neuropathy, CRPS Type I, cancer related neuropathy, and immune related neuropathy, such as chronic inflammatory demyelinating polyneuropathy (CIDP)).

I. Other Disorders

The present invention provides novel regimens for the treatment of various other disorders. Examples of other diseases and disorders include malignancy, anemia, hepatic disorders (including HBV and HCV infection, autoimmune hepatitis, fatty liver disease, hepatotoxicity, liver failure, non-alcoholic hepatitis, alcoholic hepatitis, fibrosis), nail disease, endometriosis, prostatitis, scar tissue formation, periodontal disease, spinal cord edema, pancreatitis, and gout.

### IV. Application of Novel Regimens

As described above, the present invention provides novel induction and maintenance regimens for the treatment of various diseases and disorders including but not limited to pain, spinal disorders, orthopedic disorders, inflammatory disease, immune system disease, diabetes, cardiovascular disease, and disorders of the central and peripheral nervous systems. As described herein, an induction regimen comprises a more invasive or more local site of administration than a maintenance regimen, and a lower dose per administration than the dose per administration in the maintenance regimen employed in the same patient concurrent with or following the induction regimen.

A. Pain

Treatment of any type of pain syndrome may be achieved using one or more TATs administered as an induction regimen followed by a maintenance regimen. The induction regimen may be administered via a more invasive route or more locally to the site of pain, for example, through injection or direct application (e.g., intrathecally, intradiskally, epidurally including periradicularly, perispinally). The induction regimen of one or more TATs will generally achieve a relatively rapid or immediate effect upon administration, including, for example, a reduction in severity or intensity of pain or disability, alleviation of one or more symptoms associated with pain or disability, reduction in resting pain and/or mechanically-induced pain, shortening duration of pain or disability, and reduction of pain sensitivity or sensation. A maintenance regimen of one or more TATs is typically administered once the induction regimen is administered and completed, or at or near the same time as the induction regimen. A maintenance regimen of one or more TATs is administered via a less invasive route or less locally than the induction dose, for example, intravenously, intramuscularly, subcutaneously, orally, enterally, intranasally, dermally, or by inhalation. In an alternative embodiment, the maintenance dose may be administered via a fully implanted pump or a partially implanted pump delivering the TAT by a less invasive or less local route than the induction regimen employed in the subject. For example, if the induction regimen were administered intrathecally, including by intrathecal pump, the maintenance regimen might be administered epidurally or IV, including by implantable pump. In an embodiment, the maintenance dose is administered systemically.

The dose administered for a single maintenance dose of one or more TATs is higher than the dose administered for a single induction dose of one or more TATs.

B. Preventing/Treating Spinal Disorders

Prevention or treatment of any type of spinal disorder may be achieved using one or more TATs administered as an induction regimen. The induction regimen may be administered via a more invasive route or more locally to the site of the spinal disorder, for example, intrathecally, intradiskally, epidurally, periradicularly, and perispinally. In an embodiment, the induction regimen may be administered using intradiskal/epidural, intradiskal/peridiskal, and intradiskal/peridiskal/epidural administration. The induction regimen of one or more TATs will generally achieve a rapid or immediate effect upon administration, including, for example, induction of protection from symptoms caused by a spinal disorder or invasive spinal procedure, thus preventing or postponing the development of symptoms, and induction of remission from pain caused by an established spinal disorder, for example, arthritic disorders, nerve compression disorders, disk disorders, chronic back pain disorders, stenosis, spondylolisthesis and spondylosis. A maintenance regimen of one or more TATs may be administered once the induction regimen is administered and completed, or at or near the same time as the induction regimen. The maintenance regimen is administered via a less invasive route or less locally, for example, IV, intramuscularly, SC, orally, enterally, intranasally, dermally, or by inhalation. In an alternative embodiment, the maintenance regimen may be administered via a fully implanted pump or a partially implanted pump, provided that the pump administers the drug less locally and/or less invasively than the induction regimen employed in the two stage induction-maintenance treatment. For example, following an intradiskal or intrathecal induction regimen of one or more TATs, including by an implantable pump, a maintenance regimen of one or more TATs may be administered via a fully implanted or a partially planted IV or intramuscular pump. In an embodiment, the maintenance regimen is administered systemically.

The dose administered for a single maintenance dose of one or more TATs is higher than the dose administered for a single induction dose of one or more TATs.

C. Other Orthopedic Disorders

Prevention or treatment of an orthopedic disease or disorder may be achieved using one or more TATs administered first as an induction regimen.

Treatment of an orthopedic disease or disorder may be achieved using a one or more TATs administered as an induction regimen. The induction regimen may be administered via a more invasive route or more locally to the site of the orthopedic disease or disorder, for example, using intradiskal, intrathecal, intracapsular, intramedullar, epidural, periradicular, perispinal, pericapsular, intra-articular, intra-facet, intra-cartilaginous, and intrasynovial administration. In an embodiment, the induction regimen may be administered using intradiskal/epidural, intradiskal/peridiskal, and intradiskal/peridiskal/epidural administration. In a further embodiment, the induction regimen may be administered intradiskally, intrathecally, epidurally, periradicularly, and perispinally.

In other embodiments, an induction regimen of one or more TATs may be used to improve the outcome of surgery in a patient that has undergone an orthopedic surgery, including surgery to implant a prosthetic device. In this embodiment, one or more TATs may be administered as an induction regimen prior to surgery, at the time of surgery, or at any time following surgery via a more invasive route or more locally. For example, more invasive and/or local routes of spinal delivery include, in order, intradiskally, intrathecally, epidurally, periradicularly, and perispinally, and in delivery to other joints, intracapsularly (including intra-articularly) and pericapsularly. The induction regimen may also be administered into an accommodating implanted device including, for example, a device comprising a hydrogel a device incorporating a reservoir, a synthetic bone substitute, an allograft, or a collagen sponge, any of which may also incorporate a separate therapeutic such as a bone morphogenic protein or other drug therapy.

The maintenance regimen of one or more TATs may be administered once the induction regimen is administered and completed, or at or near the same time as the induction regimen. The maintenance regimen is administered via a less invasive route or less locally, for example, periradicular, perispinal, pericapsular, in delivery to other joints, pericapsularly e.g., where the aforementioned routes of administration were not used in the induction phase), intravenously, intramuscularly, subcutaneously, orally, enterally, intranasally, dermally, or by inhalation. In an embodiment, the maintenance regimen may be administered via a fully implanted pump or a partially implanted pump. In an embodiment, the maintenance regimen is administered systemically.

The dose administered for a single maintenance dose of one or more TATs is higher than the dose administered for a single induction dose of one or more TATs.

D. Inflammatory Disease

Treatment of any type of inflammatory disorder may be achieved using one or more TATs administered as an induction regimen. The induction regimen may be administered via a more invasive route or more locally, according to the inflammatory disorder to be treated, as decided by a healthcare service provider. Potential administration sites may include, for example, the lung in inflammatory airway disorders, the gut in inflammatory bowel diseases, connective tissue in inflammatory connective tissue disease, the joint in inflammatory joint diseases, and the skin in inflammatory skin diseases. The induction regimen of one or more TATs will generally achieve rapid or immediate protection from pain associated with inflammation and will alleviate local inflammation. A maintenance regimen of one or more TATs may be administered once the induction regimen is administered and completed, or at or near the same time as the induction dose. The maintenance regimen may be administered via a less invasive route or less locally than the induction regimen, including, for example, systemically. The dose administered for a single maintenance dose of one or more TATs is higher than the dose administered for a single induction dose of one or more TATs.

In particular embodiments, chronic inflammatory airway disorders may be treated using the novel regimen of the present invention using one or more TATs administered as an induction regimen directly to the lungs and by administering the maintenance regimen systemically. For example, the induction regimen may be administered via inhalation and the maintenance regimen may be administered transdermally. Alternatively, the induction regimen may be administered via intrapulmonary or intrabronchial administration, and the maintenance regimen may be administered via inhalation.

Chronic inflammatory bowel disease may be treated using the novel regimen of the present invention using one or more TATs administered as an induction regimen directly to the gut and by administering the maintenance regimen systemically. For example, the induction regimen may be administered via intraileal administration, including during surgery, and the maintenance regimen may be administered percutaneously without surgery, including, for example, intravenously, intramuscularly, subcutaneously, orally, enterally, intranasally, dermally, or by inhalation. The induction regimen may also be administered percutaneously without surgery, including for example, intravenously, intramuscularly, subcutaneously, and the maintenance regimen may be administered systemically orally, enterally, intranasally, dermally, or by inhalation.

Chronic inflammatory connective tissue diseases and chronic inflammatory joint diseases may be treated using the novel regimen of the present invention using one or more TATs administered as an induction regimen via intracapsular, intra-articular, intrasynovial, intra-facet, and intra-cartilaginous administration. The maintenance regimen may be administered via pericapsular, IV, intramuscular, SC, oral, enteral, intranasal, transdermally, and by inhalation.

Chronic inflammatory skin disease may be treated using the novel regimen of the present invention using one or more TATs administered as an induction regimen to the skin at the site of inflammation and by administering the maintenance regimen systemically. For example, the induction regimen may be administered intravenously or subcutaneously and the maintenance regimen may be administered topically, via a transdermal patch, orally, enterally, intranasally, or by inhalation.

The dose administered for a single maintenance dose of one or more TATs is higher than the dose administered for a single induction dose of one or more TATs.

E. Immune System Disease

Treatment of any type of immunologic disorder may be achieved using one or more TATs administered as an induction regimen. The induction regimen may be administered via a more invasive route or more locally, for example, intravenously, intramuscularly, or subcutaneously. The induction regimen of one or more TATs will generally achieve a rapid or immediate effect upon administration, which may include alleviation of an inappropriate immune response, for example, suppression of autoimmunity, suppression of the immune response directed towards a transplant, suppression of an immune response directed towards a graft, suppression of graft-versus host disease, and suppression of hypersensitivity. A maintenance regimen of one or more TATs may be administered once the induction regimen is administered and completed, or at or near the same time as the induction regimen. The maintenance regimen may be administered via a less invasive route and less locally than the induction regimen, for example, orally, enterally, intranasally, dermally, or by inhalation. The maintenance regimen may be administered systemically. The dose administered for a single maintenance dose of one or more TATs is higher than the dose administered for a single induction dose of one or more TATs.

F. Metabolic Disorders

Treatment of metabolic disorders such as diabetes or obesity may be achieved using one or more TATs administered as an induction regimen. In the example of insulin dependant diabetes, the induction regimen may be administered via a more invasive route or more locally, for example, intravenously, intramuscularly, subcutaneously. The induction regimen of one or more TATs will generally achieve a rapid or immediate effect upon administration, which may include reduced B-cell destruction of the pancreatic islet cells, and alleviation of the symptoms associated with diabetic neuropathy, peripheral neuropathy, diabetic retinopathy, diabetic ulcerations, retinopathy ulcerations, diabetic macrovasculopathy, and obesity. A maintenance regimen of one or more TATs may be administered once the induction regimen is administered and completed, or at or near the same time as the induction regimen. The maintenance regimen may be administered via a less invasive route and/or less locally than the induction regimen, for example, orally, enterally, intranasally, dermally, or by inhalation. The maintenance regimen may be administered systemically.

The dose administered for a single maintenance dose of one or more TATs is higher than the dose administered for a single induction dose of one or more TATs.

Treatment of obesity may be achieved using one or more TATs administered as an induction regimen. The induction regimen may be administered via a more invasive route or more locally, for example, intravenously, intramuscularly, subcutaneously. A maintenance regimen of one or more TATs may be administered once the induction regimen is administered and completed or at or near the same time as the induction regimen. The maintenance regimen may be administered via a less invasive route or less locally than the induction regimen, for example, orally, enterally, intranasally, dermally, or by inhalation. The maintenance regimen may be administered systemically.

The dose administered for a single maintenance dose of one or more TATs is higher than the dose administered for a single induction dose of one or more TATs.

G. Cardiovascular Disease and Diseases of Endothelial Dysfunction

Treatment of cardiovascular disease may be achieved using one or more TATs administered as an induction regimen. The induction regimen may be administered via a more invasive route or more locally. A maintenance regimen of one or more TATs may be administered once the induction regimen is administered and completed, or at or near the same time as the induction regimen. The maintenance regimen may be administered via a less invasive route or less locally than the induction regimen.

The dose administered for a single maintenance dose of one or more TATs is higher than the dose administered for a single induction dose of one or more TATs.

In a particular embodiment, the induction regimen may be administered percutaneously into the myocardium or the aorta during surgery, for example, via intracardiac, intraventricular, and intracoronary administration, and the maintenance regimen may be administered percutaneously without surgery, including, for example, intravenously, intramuscularly, and subcutaneously. In this embodiment, the maintenance regimen may also be administered orally, enterally, intranasally, dermally, and by inhalation.

H. Disorders of the Central and Peripheral Nervous Systems

Treatment of central and peripheral nerve disease may be achieved using one or more TATs administered as an induction regimen. The induction regimen may be administered via a more invasive route or more locally. A maintenance regimen of one or more TATs may be administered once the induction regimen is administered and completed, or at or near the same time as the induction regimen. The maintenance regimen may be administered via a less invasive route or less locally than the induction regimen.

The dose administered for a single maintenance dose of one or more TATs is higher than the dose administered for a single induction dose of one or more TATs.

In an embodiment, central nervous system disease may be treated by administering the induction regimen via intracerebral and intracerebroventricular administration. The maintenance regimen may be administered intrathecally, epidurally, intravenously, intramuscularly, subcutaneously, orally, enterally, intranasally, dermally, and by inhalation.

In another embodiment, the induction regimen may be administered using an IV regional perfusion technique, and the maintenance dose may be administered intravenously, intramuscularly, subcutaneously, orally, enterally, intranasally, dermally, and by inhalation.

### VI. Targeted Anti-Inflammatory Therapies (TATs)

Structural Classes of TATs

TATs can be biologics (such as Abs, SMIPs, soluble receptor or coligands, or fusion proteins), polypeptides, nucleic acids, or small molecules.

Antibodies

In some embodiments of the invention, the TAT comprises an Ab, Ab fragment, or other functional equivalent thereof. Abs useful in the methods of the present invention include, without limitation, monoclonal Abs (mAbs), polyclonal Abs, Ab fragments (e.g., Fab, Fab′, F(ab′)2, Fv, Fc, etc.), chimeric Abs, mini-Abs or domain Abs (dAbs), dual specific Abs, bispecific Abs, heteroconjugate Abs, single chain Abs (SCA), single chain variable region fragments (ScFv), mutants thereof, fusion proteins comprising an Ab portion or multiple Ab portions, humanized Abs, fully human Abs, and any other modified configuration of the immunoglobulin (Ig) molecule that comprises an antigen recognition site of the required specificity, including glycosylation variants of Abs, amino acid sequence variants of Abs, and covalently modified Abs. Examples of dual specific Abs could include, but are not limited to, Abs directed to the following pairs of targets: two different antigens on the TNF molecule or TNF-R1 or R2; different chains of the TNF or TNF-R1 or R2 molecules; TNF and IL-1; TNF-R1 or R2 and TNF; TNF-R1 or R2 and IL-1; any antigen on TNF or TNF-R1 or R2 and any antigen on another IC such as IL-1, -6, -12, -15, -17, -18, -23, IFNg, GM-CSF, IL-8, MCP-1 (CCL2), and similar combinations. Methods for making such Abs are well known in the art. The Abs may be murine, rat, human, or any other origin (including chimeric, humanized, or fully human Abs). In one embodiment, the Ab recognizes one or more epitopes on an IC selected from TNF, IL-1, IL-6, IL-12, IL-15, IL-17, IL-18, IL-23, IFNg, GM-CSF, IL-8 and MCP-1 (CCL2), or recognizes one or more epitopes on an IM selected from MMP-1, 2, 3, 7, 9, 13, ADAMTS-4, 5, iNOS, NO, COX-2, and PGE2.

Antibodies also include, without limitation, agonist and antagonist Abs, as appropriate. As will be appreciated by those of skill in the art, binding affinities will vary widely between Abs, generally ranging from picomolar to micromolar levels. Methods for determining the binding affinity of an Ab are well known in the art. In some embodiments, the Ab binds an IC or IM and does not significantly bind the corresponding IC or IM from another mammalian species. In other embodiments, the Ab binds human TNF and optionally TNF from one or more non-human species.

In other embodiments, the Ab comprises a modified constant region, such as a constant region that is immunologically inert, e.g., does not trigger complement mediated lysis or stimulate Ab-dependent cell mediated cytotoxicity (ADCC) (see, e.g., U.S. Pat. No. 5,500,362). In other embodiments, the constant region is modified as described, for example, in [1]; PCT Application No. PCT/GB99/01441; and/or UK Patent Application No. 9809951.8.

Antibodies (e.g., human, humanized, mouse, chimeric) that can inhibit a protein's activity may be made by using immunogens that express the full length or a partial sequence of the protein (e.g., TNF), or cells that over expresses the protein. The Abs may be made by any method known in the art. The route and schedule of immunization of the host animal are generally in keeping with established and conventional techniques for Ab stimulation and production. Techniques for producing Abs are well known in the art including, without limitation, hybridomas, CHO cells, and other production systems; methods for primatizing or humanizing Abs and Ab fragments; methods for generating “fully human” Abs and Ab fragments; chimeric Abs; phage display technology; and recombinant technologies, such as transgenic animals and plants.

The Abs may be isolated and characterized using methods well known in the art. Abs may be isolated, for example, using conventional Ig purification procedures, such as ammonium sulfate precipitation, gel electrophoresis, dialysis, chromatography, and ultrafiltration.

SMIPs

A TAT can be a Small Modular Immuno-Pharmaceuticals (SMIP). SMIPs are single-chain polypeptides that are engineered to retain full binding and activity function of a monoclonal Ab (mAb); are approximately one-third to one-half the size of conventional therapeutic mAbs; and retain Fc-mediated effector functions. Examples of SMIP TATs for use in the present methods include TRU-015 and similar SMIPs that bind TNF or other ICs and IMs (Trubion Pharmaceuticals).

Soluble Receptors and Coligands

In some embodiments, the TAT comprises a soluble receptor or soluble co-ligand. The terms “soluble receptor”, “soluble cytokine receptor” (SCR) and “immunoadhesin” are used interchangeably to refer to soluble chimeric molecules comprising the extracellular domain of a receptor, e.g., a receptor of an IC or IM and an Ig sequence, which retains the binding specificity of the receptor and is capable of binding to the e.g., IC or IM (e.g., TNF). In one embodiment, a TNF SCR comprises a fusion of a TNF receptor amino acid sequence (or a portion thereof) from a TNF extracellular domain capable of binding TNF (in some embodiments, an amino acid sequence that substantially retains the binding specificity of the TNF receptor) and an Ig sequence. In some embodiments, the TNF receptor is a human TNF receptor sequence, and the fusion is with an Ig constant domain sequence. In other embodiments, the Ig constant domain sequence is an Ig heavy chain constant domain sequence. In other embodiments, the association of two TNF receptor-Ig heavy chain fusions (e.g., via covalent linkage by disulfide bond(s)) results in a homodimeric Ig-like structure. An Ig light chain can further be associated with one or both of the TNF receptor-Ig chimeras.

An example of a commercially available soluble receptor useful in the present invention is Enbrel® (etanercept). Enbrel® consists of recombinant human TNFR-p75-Fc fusion protein. The product is made by encoding the DNA of the soluble portion of human TNFR-p75 with the Fc portion of IgG.

Dominant-Negative Mutants

In other cases, a biologic TAT can be a dominant-negative mutant, e.g., of a polypeptide. One skilled in the art can prepare dominant-negative mutants of, e.g., the TNF receptor, such that the receptor will bind the TNF, thereby acting as a “sink” to capture TNF molecules. The dominant-negative mutant, however, will not have the normal bioactivity of the TNF receptor upon binding to TNF. The dominant negative mutant can be administered in protein form or in the form of an expression vector such that the dominant negative mutant, e.g., mutant TNF receptor, is expressed in vivo. The protein or expression vector can be administered using any means known in the art, such as intra-operatively, intraperitoneally, intravenously, intramuscularly, subcutaneously, intrathecally, intraventricularly, orally, enterally, parenterally, intranasally, dermally, or by inhalation. For example, administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Antisense and siRNA Molecules

In another embodiment, a TAT may be an antisense or siRNA molecule, e.g., to a designated IC or one of the defined polypeptides in its pathway(s), or to an IM. Nucleotide sequences of the designated ICs and the defined polypeptides in their pathways, and of the IMs are known and are readily available from publicly available databases. Exemplary sites of targeting include, but are not limited to, the initiation codon, the 5′ regulatory regions, the coding sequence and the 3′ untranslated region. In some embodiments, the oligonucleotides are about 10 to 100 nucleotides in length, about 15 to 50 nucleotides in length, about 18 to 25 nucleotides in length, or more. The oligonucleotides can comprise backbone modifications such as, for example, phosphorothioate linkages, and 2′-O sugar modifications well know in the art.

In some embodiments, the TAT is a direct IC-I or a direct IM-I comprising at least one antisense or siRNA molecule capable of inhibiting or reducing the expression of a designated IC polypeptide, a defined polypeptide in the designated polypeptide's pathway, or an IM. Alternately, expression and/or release and/or receptor expression can be decreased using gene knockdown, morpholino oligonucleotides, RNA inhibition oligonucleotides (RNAi), or ribozymes, or any other methods that are well-known in the art.

Small Molecules

In some embodiments, the TAT comprises at least one small molecule IC-I or IM-I. The small molecule can be administered using any means known in the art, including via inhalation, intra-operative administration, intraperitoneally, intravenously, intramuscularly, subcutaneously, intrathecally, intradiskally, peridiskally, epidurally, perispinally, intraventricularly, orally, enterally, parenterally, intranasally, or dermally. In general, when the TAT is a small molecule, it will be administered at the rate of 0.1 to 300 mg/kg of the weight of the patient divided into one to three or more doses. For example, in an adult patient of normal weight, the doses may range from about 1 mg to about 5 g per dose.

An exemplary small molecule for use as a TAT in the present methods is thalidomide, which is an inhibitor of TNF production. The term “thalidomide” refers to an anti-inflammatory agent sold under the trademark THALOMID® (Celgene), and all pharmaceutically acceptable prodrugs, salts, solvate, clathrates and derivatives thereof. The term “derivative” means a compound or chemical moiety wherein the degree of saturation of at least one bond has been changed (e.g., a single bond has been changed to a double or triple bond) or wherein at least one hydrogen atom is replaced with a different atom or a chemical moiety. Examples of different atoms and chemical moieties include, but are not limited to, halogen, oxygen, nitrogen, sulfur, hydroxy, methoxy, alkyl, amine, amide, ketone, and aldehyde. Exemplary thalidomide derivatives include, without limitation, taglutimide, supidimide, compounds disclosed in WO 94/20085, 6-alkyl-2-[3′- or 4′-nitrophthalimido]-glutarimides and 6-alkyl-3-phenylglutarimides [see e.g., (2)]; and lenalidomide, a derivative of thalidomide sold under the trademark REVLIMID® (Celgene), also known as CC-5013, which is described, for example, in [3].

Other small molecules that possess TAT, particularly TNF-I, activity include, without limitation, tetracyclines (e.g., tetracycline, doxycycline, lymecycline, oxytetracycline, minocycline), chemically modified tetracyclines (e.g., dedimethylamino-tetracycline), hydroxamic acid compounds, carbocyclic acids and derivatives, lazaroids, pentoxifylline, napthopyrans, amrinone, pimobendan, vesnarinone, phosphodiesterase inhibitors, and small molecule inhibitors of kinases. Small molecule kinase inhibitors include, without limitation, small molecule inhibitors of p38MAPK, COT, MK2, P13K, IKKa,b,g, MEKK1,2,3, IRAK1,4 and Akt kinase. See also US Pat. Publications 2006/0046961; 2006/0046960; and 2006/0253100 for examples of small molecule inhibitors for use in the present methods.

Biogenerics, Biosimilars, Follow On Biologics, and Follow-On Proteins

The TAT, including a direct TNF-I, could also be a biosimilar, biogeneric, follow-on biologics, or follow-on protein version of a currently contemplated TAT, including a direct TNF-I. For example, once the patents covering Enbrel® (etanercept) expire, other manufacturers will likely produce molecules similar or identical to etanercept, by manufacturing processes that are substantially similar or the same, or different from, those used to manufacture Enbrel®. Their objective would be to make, offer to sell, and sell therapeutics similar or identical in structure and activity to Enbrel® (etanercept). Such molecules are generally referred to as biogenerics, generic biologics, biosimilars, follow on biologics, and follow on proteins, depending on details of the molecule, the manufacturing process and the regulatory pathway. In certain instances, the new product might differ by one or a few amino acids, which might be purported to improve the manufacturing efficiency or the therapeutic efficacy. In all such instances, these molecules are viewed as substantially the same as, or the same as currently contemplated TATs, including direct TNF-Is.

Targets And Examples of TATs

TATs for use in the invention can be IC-Is or IM-Is. In inflammation, each IC has a unique profile of biological activity, often representing multiple distinct activities. These activities are mediated by interaction of the cytokines with their receptors on a variety of inflammatory and tissue cell types. The cellular effects of ICs are mediated by intracellular signaling pathways, many of which result in activation of transcription factors which in turn activate transcription of genes encoding IC, proteinacious IM, and other proteins.

IC-Is

A TAT can be an inhibitor of one of the following IC designated polypeptides or one of the defined polypeptides in their pathways, as described further herein: TNF, IL-1, IL-6, IL-12, IL-15, IL-17, IL-18, IL-23, IFNg, GM-CSF, IL-8, MCP-1 (CCL2).

TNF-Is, Including Direct TNF-Is

TNF is produced primarily by stimulated macrophages, T cells and mast cells by cleavage of Pro TNF by TNF alpha converting enzyme (TACE). TNF induces the production of IL-1, IL-6, IL-8, IL-17, GM-CSF, PGE₂ and NO from macrophages, thus placing TNF near the top of a proinflammatory cascade. TNF also induces the production of the matrix-degrading proteolytic enzymes, MMPs and ADAMTSs, from chondrocytes, fibroblasts and other cells.

The biological effects of TNF are mediated via binding of TNF to either of two receptors, TNFR1 and TNFR2. Several signaling pathways may be activated (FIG. 2). One pathway leads to NFκB activation and is mediated by signaling proteins, including TRADD, RIP, TRAF2, MEKK-3, IKKα,β,γ, IKB-α, p50, Rel A and proteasomes. An alternative pathway to NFκB activation involves PI3K, Akt and COT prior to the IKK complex. Another pathway leads to apoptosis of the cell and is mediated by TRADD, FADD and Caspase-3 and 8 and blocked by FLICE. A fourth pathway leads to AP-1 activation and involves Rac-1, MEKK-1, 2, MKK3,4,6,7, JNK, p38MAPK and MK2. The term “TNF inhibitor” or “TNF-I” refers to any molecule which can block, suppress or reduce gene expression, protein production and processing, release, and/or biological activity of TNF, its biological receptor, coreceptor, or coligand, or a defined polypeptide in the TNF pathways (FIG. 2). Thus, examples of TNF-Is include inhibitors of any of the following polypeptides: ProTNF, TNF, TNFR1 and TNFR2, caspase 3, caspase 8, FADD, NFκB, IκB-alpha, TACE, TRADD, RIP, TRAF2, MEKK3, P13K, Akt, COT, IKKalpha, IKKbeta, IKKgamma, p50, RelA, TRAF6, FLICE, Rac-1, MEKK-1, 2, MKK3,4,6,7, JNK, p38MAPK, MK2, JUN and FOS.

A TNF-I can inhibit either or both of the two receptors TNFR1 (TNF receptor type 1) and TNFR2 (TNF receptor type 2). Some TNF-Is can inhibit a cysteine aspartase protease, such as caspase 3 or caspase 8; or can inhibit FADD; or can inhibit TRAF2. Some TNF-Is can inhibit IκB, a protein which inhibits the cell survival pathway mediator protein Nuclear factor-kappa B (NFκB). Some TNF-Is may inhibit NFκB. Examples of NFκB-Is include sulfasalazine, sulindac, clonidine, helenalin, wedelolactone, pyrollidinedithiocarbamate (PDTC), IKK-2 inhibitors, IKK inhibitors, and others, e.g., those set forth in US Pat. Publication 2006/0253100. Some TNF-Is may inhibit TNF converting enzyme (TACE), a metalloproteinase that processes pro-TNF into its mature, soluble form for release. Drugs that selectively inhibit TACE, and thereby effectively block the processing and release of mature TNF, show anti-inflammatory effects and significant decreases in cytokine production in vitro and in vivo.

Preferred inhibitors for use in the present methods are direct TNF-Is. Examples of direct TNF-Is useful in the practice of the present invention include, without limitation, the marketed products etanercept, infliximab, adalimumab and certolizumab pegol (Cimzia®; peg-antiTNF alpha Ab fragment) (formerly CDP 870; UCB/Celltech, now Nektar). Examples of direct TNF-Is currently in clinical development include the fully human anti-TNF mAb CNTO-148 (golimumab, Centocor/J&J), and the anti-TNF mAb AME-527 (Applied Molecular Evolution/Eli Lilly).

Examples of direct TNF-Is currently in pre-clinical development include the fully human anti-TNF mAb ABX-10131 (Abgenix/Amgen); several Ab fragments in development by companies such as Domantis/Peptech and AbLynx; and the SMIP TRU-015 being developed by Trubion Pharmaceuticals. Other examples of direct TNF-Is include ABX-10131; polyclonal anti-TNF Abs such as made by therapeutic human polyclonals (THP); anti-TNF polyclonal anti-serum such as that made by Genzyme; pegylated soluble TNF receptor Type I (pegsunercept/PEGs TNF-R1); Onercept (recombinant TNF binding protein (r-TBP-1)); trimerized TNF antagonist; dominant negative TNF proteins such as Xencor's dominant negative TNF-I; modified sTNR1 (Biovation); Humicade® (CDP-570); and PN0621 (mini-Abs against TNF).

IL-1 Inhibitors, Including Direct IL-1 Inhibitors

IL-1 (a term which includes both IL-1α and IL-1β forms) is produced by processing of the precursor proteins, Pro IL-1α and Pro IL-1β, in an intracellular “inflammasome” involving P2X7, NALP3, ASC and Caspase-1 (FIG. 2). The predominant circulating form of IL-1 is IL-1β, whereas IL-1α primarily remains cell-membrane associated. IL-1 binds to its receptor, IL-1R1 and that complex then binds to IL-1RAcP (accessory protein), which enables signal transduction. The biological effects of IL-1 are mediated by two pathways (FIG. 2). One pathway leads to NFkB activation and involves MyD88, TIRAP, IRAK1,4, TRAF6 and the IKK complex shared by the TNF pathway. The other pathway leads to AP-1 activation and links the MyD88/TIRAP/IRAK-1,4 complex with Rac-1 and downstream elements shared by TNF.

The term “IL-1 inhibitor” or “IL-1-I” refers to any molecule which can block, suppress or reduce gene expression, protein production and processing, release, and/or biological activity of IL-1, its biological receptor, coreceptor, or coligand, or a defined polypeptide in the IL-1 pathways shown in FIG. 2. Examples of IL-1-Is include inhibitors of any of the following polypeptides: IL-1 alpha, IL-1 beta, Pro IL-1, P2X7, NALP3, ASC, Caspase-1, IL-1R1, IL-1RAcP, IRAK1, MyD88, TIRAP, IRAK4, TRAF6, Rac-1, MEKK-1, MEKK-2, MEKK-4, MEKK-7, JNK, JUN, FOS, MK2, p38 MAP kinase, MEKK-3, MEKK-6, AP-1, IKKalpha, -beta, or -gamma; IkB-alpha, p50, Rel A and NFκB.

Examples of IL-1-I are VX740 and VX765, small molecule caspase-1 inhibitors previously in clinical development for rheumatoid arthritis (Vertex). Some IL-1-Is can inhibit p38 kinase (p38 MAP kinase). Over 100 p38 kinase inhibitors have been identified, many of which compete with ATP and are able to bind both active and inactive (phosphorylated and unphosphorylated) forms of the MAP kinase. In other cases, tyrosine-specific phosphatases can inhibit p38 MAPK by dephosphorylating the kinase at key positions. Treatment of arthritic animal models with synthetic p38 inhibitors suggests that p38 inhibition can produce protective anti-inflammatory effects in vivo. Small molecule inhibitors of p38 MAPK have demonstrated a broad range of anti-inflammatory effects mediated by changes in cytokine production. Exemplary small molecule p38 kinase inhibitors are described in US 2005/0025765.

A direct IL-1-I can be an inhibitor of an IL-1 receptor. Interleukin-1 receptor antagonist (IL-1 Ra) is a naturally occurring molecule which reduces the biologic effects of interleukin-1 by interfering with the binding of IL-1 to its receptor (IL-1 R1, interleukin-1 type I receptor). Kineret® (Amgen) is a recombinant form of IL-1 Ra which is FDA-approved for treating rheumatoid arthritis. Another example of a direct IL-1-I is AMG108, a mAb directed to IL-1R, currently in clinical development in rheumatoid arthritis (Amgen). AMG719 (sIL-1R2, Amgen), and IL-1 Trap (Regeneron), are also all direct inhibitors of IL-1. Another example of a direct IL-1-I is ACZ885 (a fully human anti-interleukin-1beta (anti-IL-1beta) mAb) in clinical development for Muckle-Wells Syndrome (Novartis).

IL-6 Inhibitors, Including Direct IL-6 Inhibitors

The effects of IL-6 are mediated by binding of IL-6 to IL-6Rα, either in soluble or membrane-bound form. The IL-6/IL-6Rα complex then binds to gp130 in the cell membrane to initiate signaling. Key proteins involved in the IL-6 pathway are JAK1, STAT1 and STAT3. The term “IL-6 inhibitor” or “IL-6-I” refers to any molecule which can block, suppress or reduce gene expression, protein production and processing, release, and/or biological activity of IL-6, its biological receptor, coreceptor, or coligand, or a defined polypeptide in the IL-6 pathway. Defined polypeptides in the IL-6 pathway are IL6Ralpha, gp130, JAK1, STAT1, and STAT3. An example of a direct IL-6-I is the humanized anti-IL6 receptor mAb Tocilizumab (Actemra®, Chugai). Another example of a direct IL-6-I is AMG 220, an Avimer™ protein, which binds to IL-6. AMG 220 is being studied in Crohn's disease patients. Another example of a direct IL-6-I is CNTO 328 (Anti IL-6 MAb) in clinical development for refractory multiple myeloma (Centocor). Another example of a direct IL-6-I is C326, an Avimer™ protein inhibitor of IL-6, in Crohn's Disease (Avidia).

IL-8 Inhibitors, Including Direct IL-8 Inhibitors

IL-8 is a chemokine also known as CXCL8. IL-8 mediates its activities through either of two receptors, CXCR1 and CXCR2, which are also receptors for other chemokines. Key proteins involved in the IL-8 pathway are PKC, PLC, PLD, Ras, rho and PI3K. The term “IL-8 inhibitor” or “IL-8-I” refers to any molecule which can block, suppress or reduce gene expression, protein production and processing, release, and/or biological activity of IL-8, its biological receptor, coreceptor, or coligand, or a defined polypeptide in the IL-8 pathway. Defined polypeptides in the IL-8 pathway are CXCR1, CXCR2, PKC, PLC, PLD, Ras rho and PI3K. An example of a direct IL-8-I is ABX-IL8, a fully human anti-IL-8 mAb previously in clinical development for psoriasis, COPD and chronic bronchitis (Abgenix).

IL-12 Inhibitors, Including Direct IL-12 Inhibitors

IL-12 is a heterodimer comprised of IL-12p40 and IL-12p35 chains, the former also being part of the IL-23 molecule. IL-12 mediates its activities through a heterdimeric receptor comprised of IL-12Rβ1 and IL-12Rβ2, again the former being part of the IL-23R. Key proteins involved in the IL-12 pathway include TYK2, JAK2 and STAT4. The term “IL-12 inhibitor” or “IL-12-I” refers to any molecule which can block, suppress or reduce gene expression, protein production and processing, release, and/or biological activity of IL-12, its biological receptor, coreceptor, or coligand, or a defined polypeptide in the IL-12 pathway. Defined polypeptides in the IL-12 pathway are IL-12p40, IL-12p35, IL-12Rβ1, IL-12Rβ2, TYK2, JAK2 and STAT4. An example of an IL-12-I is the small molecule STA-5326 Meslylate in clinical development to treat gut inflammation (Synta). An example of a direct IL-12-I is ABT-874, a human mAb directed against IL-12p40, in clinical development for psoriasis and other inflammatory diseases (Abbott). Another example of a direct IL-12-I is CNTO 1275 a human mAb directed against IL-12p40, in clinical development for psoriasis and other inflammatory diseases (Centocor).

IL-15 Inhibitors, Including Direct IL-15 Inhibitors

IL-15 mediates its activities by binding to a heterotrimeric receptor comprised of an IL-15Ra chain, an IL-2/15Rβ chain and the “common γ chain” γc. Key proteins involved in the IL-15 pathway include JAK1,3 and STAT3,5. The term “IL-15 inhibitor” or “IL-15-I” refers to any molecule which can block, suppress or reduce gene expression, protein production and processing, release, and/or biological activity of IL-15, its biological receptor, coreceptor, or coligand, or a defined polypeptide in the IL-15 pathway. Defined polypeptides involved in the IL-15 pathway are IL-15Ralpha, IL-2/IL-15Rbeta, the common gamma chain “gamma-c”, JAK1, JAK3, STAT3 and STAT5. An example of a direct IL-15-I is AMG 714, a fully human mAb (formerly called HuMAX15) directed against IL-15 in clinical development by Amgen/Genmab.

IL-17 Inhibitors, Including Direct IL-17 Inhibitors

IL-17 mediates its effects via an IL-17R that is expressed on virtually all cell types. Key proteins involved in the IL-17 pathway include TRAF6 and the same downstream IKK complex leading to NFκB activation as in IL-1 pathway. The term “IL-17 inhibitor” or “IL-17-I” refers to any molecule which can block, suppress or reduce gene expression, protein production and processing, release, and/or biological activity of IL-17, its biological receptor, coreceptor, or coligand, or a defined polypeptide in the IL-17 pathway. Defined polypeptides in the IL-17 pathway are IL-17R, MyD88, TIRAP, IRAK1, IRAK4, TRAF6, IKKalpha, IKKbeta, IKKgamma, IkappaB-alpha, p50, Rel A, Proteasome, NFκB and FLICE.

IL-18 Inhibitors, Including Direct IL-18 Inhibitors

IL-18 binds to a 4-chain receptor complex comprised of IL-18Rα, IL-I8RP, IL-1RAcP and a pathway chain. A naturally-occurring antagonist of IL-18 called IL-18BP blocks the binding of IL-18 to its receptor. Key proteins involved in the IL-18 pathway include MyD88 and all the downstream elements via TRAF6 leading to NFκB activation as in IL-1 pathway. The term “IL-18 inhibitor” or “IL-18-I” refers to any molecule which can block, suppress or reduce gene expression, protein production and processing, release, and/or biological activity of IL-18, its biological receptor, coreceptor, or coligand, or a defined polypeptide in the IL-18 pathway. Defined polypeptides in the IL-18 pathway are Pro IL-18, P2X7, NALP3, ASC, Caspase-1, IL-18, IL-18Ralpha, IL-18Rbeta, IL-1RAcP, IL-18R signaling chain, IL-18BP, MyD88, TIRAP, IRAK1, IRAK4, TRAF6, IKKalpha, IKKbeta, IKKgamma, IkappaB-alpha, p50, Rel A, Proteasome, NFκB, FLICE, Rac-1, MEKK-1, MEKK-2, MKK3, MKK4, MKK6, MKK7, JNK, p38MAPK, MK2, JUN, FOS and AP-1.

IL-23 Inhibitors, Including Direct IL-23 Inhibitors

IL-23 is a heterodimer of IL-12p40 and IL-23p19 chains and binds to a heterodimeric IL-23 receptor comprised of IL-12Rβ1 and IL-23R. Key proteins involved in the IL-23 pathway include TYK2, JAK2 and STAT3. The term “IL-23 inhibitor” or “IL-23-I” refers to any molecule which can block, suppress or reduce gene expression, protein production and processing, release, and/or biological activity of IL-23, its biological receptor, coreceptor, or coligand, or a defined polypeptide in the IL-23 pathway. Defined polypeptides in the IL-23 pathway are IL-12p40, IL-23p19, IL-12Rβ1, IL-23R, TYK2, JAK2 and STAT3. An example of a direct IL-23-I is ABT-874, a human mAb directed against IL-12p40, in clinical development for psoriasis and other inflammatory diseases (Abbott). Another example of a direct IL-23-I is CNTO 1275, a human mAb directed against IL-12p40, in clinical development for psoriasis and other inflammatory diseases (Centocor).

IFNγ Inhibitors, Including Direct IFNγ Inhibitors

The effects of IFNγ are mediated by homodimers of IFNγ binding to a receptor comprised of an IFNγ Rα ligand-binding chain and an IFNγ Rβ signaling chain. Key proteins involved in the IFNγ pathway include JAK1, JAK2 and STAT1. The term “IFNγ inhibitor” or “IFNγ-I” refers to any molecule which can block, suppress or reduce gene expression, protein production and processing, release, and/or biological activity of IFNγ, its biological receptor, coreceptor, or coligand, or a defined polypeptide in the IFNγ pathway. Defined polypeptides in the IFNγ pathway are IFNγRα, IFNγRβ, JAK1, JAK2 and STAT3.

GM-CSF Inhibitors, Including Direct GM-CSF Inhibitors

GM-CSF binds to a heterodimeric receptor comprised of GMRα and a common β subunit, βc. Key proteins involved in the GM-CSF pathway include JAK2, STAT5, SHP-2, RAS and Raf-1. The term “GM-CSF inhibitor” or “GM-CSF-I” refers to any molecule which can block, suppress or reduce gene expression, protein production and processing, release, and/or biological activity of GM-CSF, its biological receptor, coreceptor, or coligand, or a defined polypeptide in the GM-CSF pathway. Defined polypeptides in the GM-CSF pathway are GMRalpha/Beta-c, JAK2, STAT5, SHP-2, RAS and Raf-1.

MCP-1 Inhibitors, Including Direct MCP-1 Inhibitors

MCP-1 is a chemokine also known as CCL2. MCP-1 mediates its activities by binding to a single receptor, CCR2. Key proteins involved in the MCP-1 pathway include PKC and the same IKK complex and downstream elements as in TNF/IL-1 pathway leading to NFκB activation. The term “MCP-1 inhibitor” or “MCP-1-I” refers to any molecule which can block, suppress or reduce gene expression, protein production and processing, release, and/or biological activity of MCP-1, its biological receptor, coreceptor, or coligand, or a defined polypeptide in the MCP-1 pathway. Defined polypeptides in the MCP-1 pathway are CCR2, PKC, IKKalpha, IKKbeta, IKKgamma, IkappaB-alpha, p50, Rel A, Proteasome, NFκB and FLICE. An example of a direct MCP-11 is ID9, a mAb directed against the MCP-1 receptor CCR2 (Millenium).

IM-Is

A TAT can be an inhibitor of one of the following IMs: MMP-1,2,3,7,9,13; ADAMTS-4, 5; iNOS, NO, COX-2, and PGE2.

MMP Inhibitors, Including Direct MMP Inhibitors

The term “MMP-1, 2, 3, 7, 9, 13 inhibitor” or “MMP-1-I, 2-I, 3-I, 7-I, 9-I, 13-I” refers to any molecule which can block, suppress or reduce gene expression, protein production and processing, release, and/or biological activity of the respective MMP-1, 2, 3, 7, 9, or 13 polypeptide, or the biological receptor, coreceptor, or coligand of the same. Examples of broad-spectrum (nonspecific) direct MMP-Is include the small molecule compounds marimastat and batimastat, previously in clinical development (British Biotech, Inc). An example of a class of direct MMP-13-I with selectivity relative to other MMPs is the small molecule genus of 3-hydroxy-4-arylsulfonyltetrahydropyranyl-3-hydroxamic acids previously in clinical development (Pfizer).

An example of a direct MMP-2-I and direct MMP-9-I is XL784, a relatively selective small molecule compound in clinical development (Exelixis).

iNOS Inhibitors, Including Direct iNOS Inhibitors

The term “iNOS inhibitor” or “iNOS-I” refers to any molecule which can block, suppress or reduce gene expression, protein production and processing, release, and/or biological activity of iNOS, or its biological receptor, coreceptor, or coligand. An example of a direct iNOS-I is GW274150, a small molecule compound in clinical development for rheumatoid arthritis and migraine (GSK). Another example of a direct iNOS-I is aminoguanidine, a small molecule compound evaluated in clinical endotoxemia (Radboud University). Another example of a direct iNOS-I is SC-51, a small molecule compound in clinical development for asthma (Pfizer).

COX-2 Inhibitors, Including Direct COX-2 Inhibitors

The term “COX-2 inhibitor” or “COX-2-I” refers to any molecule which can block, suppress or reduce gene expression, protein production and processing, release, and/or biological activity of COX-2, or its biological receptor, coreceptor, or coligand. Examples of direct COX-2-I are celecoxib (Celebrex®, Pfizer) and rofecoxib (Vioxx®, Merck), small molecule compounds for treatment of inflammation and pain.

Combination Therapies

Multiple TAT Inhibitors, Including Multiple TNF-I

The present disclosure also contemplates the use of multiple TATs in the methods described herein. The combination of different TATs that have specificity for different points in a pathway, e.g., a TNF pathway, or different points in two or more different pathways, may be more efficient than the use of a single TAT. For instance, TNF itself may be inhibited at multiple points and by targeting various mechanisms in the TNF pathways. Potential inhibition points include TNF transcriptional synthesis, translation, or shedding mediated by MMPs. TNF and other similar bioactive substances are first produced in an inactive form and transported to the cell membrane. Upon activation, the active part of the pro-TNF is cleaved and released. This process is called shedding and may be initiated by one or more MPs. TNF may also be inhibited after its release, either by Abs (e.g., by infliximab, adalimumab, or CDP-870) or soluble receptors (e.g. etanercept).

The combination of two or more drugs that act through different mechanisms may therefore induce a more efficient inhibition of an IC or IM pathway than the use of one single drug. In one embodiment, a direct TNF-I is used in combination with a second direct TNF-I, or with a non-specific TNF-I or an inhibitor of a different IC or IM. In another embodiment, a direct TNF-I is used in combination with an NFκB-I such as sulfasalazine, sulindac, clonidine, helenalin, wedelolactone, pyrollidinedithiocarbamate (PDTC), IKK-2 inhibitors, IKK inhibitors, and others, e.g., those set forth in US Pat. Publication 2006/0253100.

Supplemental Active Ingredients

A TAT, e.g., TNF-T, may be administered in combination with other drugs or compounds, provided that these other drugs or compounds do not significantly reduce or eliminate the desired results according to the present invention, e.g., the effect on a IC or IM of interest such as TNF. Specific methods of the invention comprise administering a TAT in combination with an SAI. The SAI can be any TAT. Further, the SAI can be a therapeutic agent capable, for example, of relieving pain, providing a sedative effect or an antineuralgic effect, or ensuring patient comfort. Examples of the SAIs include, but are not limited to, opioid analgesics, non-narcotic analgesics, anti-inflammatories, cox-2 inhibitors, α-adrenergic receptor agonists or antagonists, ketamine, anesthetic agents, NMDA antagonists, immunomodulatory agents, immunosuppressive agents, antidepressants, anticonvulsants, antihypertensives, anxiolytics, calcium channel blockers, muscle relaxants, corticosteroids, hyperbaric oxygen, neuroprotectants, antibiotics, other therapeutics known to relieve pain, and pharmaceutically acceptable salts, solvates, hydrates, stereoisomers, clathrates, prodrugs and pharmacologically active metabolites of any of the foregoing therapeutic agents.

In another embodiment, the supplement active ingredient is a non-steroidal anti-inflammatory drug (NSAID), corticosteroid, slow acting antirheumatic drug (SAIRD), disease modifying antirheumatic drug (DMARD), short-acting local anesthetic (SALA), or long-acting local anesthetic (LALA). In yet another embodiment, the SAI is a propionic acid derivative, such as ibuprofen or naproxen. Structurally related propionic acid derivatives having similar analgesic and anti-inflammatory properties are also intended to be encompassed by this group. In another embodiment, the SAI is an acetic acid derivative, for example alclofenac, diclofenac sodium, or sulindac. Structurally related acetic acid derivatives having similar analgesic and anti-inflammatory properties are also intended to be encompassed by this group. The SAI may also be a fenamic acid derivative such as, without limitation, enfenamic acid, etofenamate, or flufenamic acid. Structurally related fenamic acid derivatives having similar analgesic and anti-inflammatory properties are also intended to be encompassed by this group.

In other embodiments, the SAI is a carboxylic acid derivative, a butyric acid derivative, or oxicam, a pyrazole, or a pyrazolon.

In another embodiment, the SAI is an antibiotic. Exemplary antibiotics include, without limitation, sulfa drugs (e.g., sulfanilamide), folic acid analogs (e.g., trimethoprim), beta-lactams (e.g., penacillin, cephalosporins), aminoglycosides (e.g., stretomycin, kanamycin, neomycin, gentamycin), tetracyclines (e.g., chlorotetracycline, oxytetracycline, and doxycycline), macrolides (e.g., erythromycin, azithromycin, and clarithromycin), lincosamides (e.g., clindamycin), streptogramins (e.g., quinupristin and dalfopristin), fluoroquinolones (e.g., ciprofloxacin, levofloxacin, and moxifloxacin), polypeptides (e.g., polymixins), rifampin, mupirocin, cycloserine, aminocyclitol (e.g., spectinomycin), glycopeptides (e.g., vancomycin), and oxazolidinones (e.g., linezolid).

In another embodiment, the SAI is capable of providing a neuroprotective effect. In addition to TNF, other examples of neuroprotective agents include, without limitation, erythropoietin (Epo), Epo derivatives or mimetics, and other compounds that stabilize or protect neurons from injury. Epo and its derivatives or mimetics might offer particular advantages, or otherwise be particularly appropriate, to patients undergoing surgery. Usage of Epo or Epo-mimetics as neuroprotectants may be limited by the difficulty in separating the neuroprotective effects of Epo from the erythrogenic effects. However, a particular setting in which such erythrogenic “side effects” are acceptable is in patients about to undergo surgery, in whom a moderate and temporary increase in hematocrit may be desirable. Thus, in peri-operative usage to improve surgical outcome, Epo may offer surprising advantages as a neuroprotectant.

The SAI could also be ozone as delivered to the spinal structure by ozone therapy [4].

### VII. Kits

The present invention also includes kits. In one embodiment, the kits of the present invention comprise one or more induction doses of a TAT of the present invention, and one or more maintenance doses of a TAT of the present invention. The composition, shape, and type of dosage form for the induction regimen and maintenance regimen may vary depending on the disease or disorder to be treated. For example, the induction regimen and maintenance regimen may be parenteral dosage forms, oral dosage forms, delayed or controlled release dosage forms, topical and mucosal dosage forms, including any combination thereof.

In a particular embodiment, a kit of the present invention can contain one or more of the following in a package or container: (1) an induction regimen of one or more TATs and a maintenance regimen of one or more TATs; (2) one or more pharmaceutically acceptable adjuvants or excipients (e.g., a pharmaceutically acceptable salt, solvate, hydrate, stereoisomer, and clathrate); (3) one or more vehicles for administration of the induction regimen of one or more TATs, such as one or more syringes, a catheter, a pump, a hydrogel, and a depot formulation form of administration; (4) one or more vehicles for administration of the maintenance regimen of one or more TATs, such as one or more syringes, patches, a catheter, a pump, a hydrogel, and a depot formulation form of administration; (5) one or more additional bioactive agents for concurrent or sequential administration with the induction regimen of one or more TATs, such as SAIs; (6) one or more additional bioactive agents for concurrent or sequential administration with the maintenance regimen of one or more TATs, such as supplemental active ingredients; (7) instructions for administration; (8) a related diagnostic instrument, reagent, test or assay, such as, for example, a catheter, needle or pump used in a diskogram, that could also be used to administer the TAT; or a separate diagnostic reagent or test that would help guide the choice of TAT therapy. Embodiments in which two or more, including all, of the components (1)-(7), are found in the same container can also be used.

When a kit is supplied, the different components of the compositions included can be packaged in separate containers and admixed immediately before use. Such packaging of the components separately can permit long term storage without losing the active components' functions. When more than one bioactive agent is included in a particular kit, the bioactive agents may be (1) packaged separately and admixed separately with appropriate (similar of different, but compatible) adjuvants or excipients immediately before use, (2) packaged together and admixed together immediately before use, or (3) packaged separately and admixed together immediately before use. If the chosen compounds will remain stable after admixing, the compounds may be admixed at a time before use other than immediately before use, including, for example, minutes, hours, days, months, years, and at the time of manufacture.

The compositions included in particular kits of the present invention can be supplied in containers of any sort such that the life of the different components are optimally preserved and are not adsorbed or altered by the materials of the container. Suitable materials for these containers may include, for example, glass, organic polymers (e.g., polycarbonate and polystyrene), ceramic, metal (e.g., aluminum), an alloy, or any other material typically employed to hold similar reagents. Exemplary containers may include, without limitation, test tubes, vials, flasks, bottles, syringes, and the like. In a particular embodiment, the different components of the compositions may be contained in a pre-filled syringe, for example, the induction regimen of one or more TATs may be contained in a pre-filled syringe.

As stated above, the kits of the present invention may also be supplied with instructional materials. These instructions may be printed and/or may be supplied, without limitation, as an electronic-readable medium, such as a floppy disc, a CD-ROM, a DVD, a Zip disc, a video cassette, an audiotape, and a flash memory device. Alternatively, instructions may be published on a internet web site or may be distributed to the user as an electronic mail.

The kits of the present invention may include kits for the treatment of a disease or disorder, including diseases or disorders in which IC or IM activity is implicated as causing, contributing to, or perpetuating the pathophysiology of the disease or disorder. In an embodiment, a kit may include, for example, a syringe, such as a pre-filled syringe containing an induction regimen of one or more TATs. This syringe may be used to administer a TAT invasively and/or more locally, including, for example, intracerebral administration. This kit may also include, for example, a syringe, such as a pre-filled syringe containing a maintenance regimen of one or more TATs. This syringe may be used to administer a TAT less invasively and less locally, for example via IV administration. The kit may also contain other devices or apparatus that may be used to administer the induction regimen and the maintenance regimen according to any of the administration techniques described herein. Examples of such devices include, but are not limited to syringes, needles, catheters, drip bags, patches, and inhalers. In some embodiments, the kit may include, for example, some or all of the necessary syringes needles, catheters, and other disposable equipment useful for epidural, including transforaminal placement, either with or without fluoroscopic guidance, for intradiskal or peridiskal administration, or for intradiskal/peridiskal, intradiskal/epidural, peridiskal/epidural, or intradiskal/peridiskal/epidural injection. Similarly, the kit may contain the necessary syringes, needles, and tubes for IV, intramuscular, and SC administration.

### VIII. Determining the Efficacy of Treatment

Methods for preventing or treating diseases or disorders in accordance with the present invention may be evaluated for efficacy using any one or more of a number of well known and art-recognized methods and will depend on the disease or disorder treated. For example, assessment of pain may be performed based on objective measures, such as observation of behavior in response to stimuli, facial expressions and the like. Assessment may also be based on subjective measures, such as patient characterization of pain using various pain scales. See, e.g., [5] and [6].

Pain relief may also be characterized by time course of relief, by objectively or subjectively assessing pain at 1, 2, or a few hours (e.g., 12-18 hours), and/or at days, weeks, or months following surgery.

Assays frequently used include, without limitation, daily spontaneous leg pain (DSLP) scores, Visual analog scale (VAS) scores, maximum daily leg pain rating, pain relief rating, average daily back or neck pain rating, daily pain at night (pain at rest), days in hospital, modified neurological examination, Oswestry low back pain disability questionnaire, SF36 questionnaire, number of sick leave days taken due to back or neck pain, sleep interference, subject's global impression of change, and the investigator's global impression of change.

Determining a level or duration of pain in a subject can be done using standard methods known to those having ordinary skill in the art. In some case, the results of any of the assessment methods can be compared with a similar assessment performed prior to administration of the TAT. Multiple assessments during a course of TAT administration are also contemplated, e.g., 2, 3, 4, 5, 6 or more temporally separate assessments. Any suitable amount of time between assessments can occur, and can be determined by one having ordinary skill in the art. In some embodiments, from about 5 days to about 2 months, or any time therebetween, elapses between assessments.

Pain relief may also be characterized by time course of relief. Accordingly, in some embodiments, pain relief is subjectively or objectively observed after 1, 2, or a few hours. In another embodiment, pain relief is subjectively or objectively observed at 24, 36, 48, 60 72 or more hours. Other non-limiting methods for assessing efficacy of treatment are described below.

The efficacy of the regimens of the present invention can be tested using animal models well-known in the art, or in human patients, as described below.

### X. Advantages of the Novel Regimen

In an embodiment, the present invention provides a novel regimen that may provide targeted treatment for a disease or disorder. In an embodiment, the induction regimen may provide a targeted and rapid improvement in a disease or disorder using a more invasive or more local route of administration and a lower dose per administration than the maintenance regimen. The maintenance regimen may provide a continuing and/or long-term improvement in a disease or disorder using a less invasive or less local route of administration and a higher dose per administration.

In an embodiment, the present invention may eliminate the need for other long-term and/or invasive treatments.

In an embodiment, an improved outcome that may be associated with the novel regimen may include a substantial improvement in a disease or disorder, including, for example, a beneficial reductions in symptoms, injury or pain from a spinal disorder, an orthopedic disorder, an inflammatory disease, an immune system disease, a metabolic disorder, a cardiovascular disease, a disease of endothelial dysfunction, and a disorder of the central and peripheral nervous system.

In an embodiment, the improved outcome may include, for example, a substantial reduction of a subject's pain, reduced sciatica, a beneficial reduction in inflammation (including cell mediated inflammation), reduced disability, a substantially reduced post-operative disorder (including post-operative ileus), a reduction in a subject's recovery time (including following a surgical procedure, a non-surgical procedure, and injury), improvement of a spinal disorder, prevention of a spinal disorder, improvement of an orthopedic disorder, an increased tolerance to physical therapy, reduced neurological degeneration, a reduced neurological disorder (including Alzheimer's disease and Parkinson's disease), and normalized insulin production. A subject may also regain mobility earlier than would be expected for a subject with a similar physical status that has not been treated with the novel regimen, for example, a subject with a similar body mass index (BMI), a subject with a similar disability, a subject with a similar body habitus, a subject that has undergone a similar degree of surgery, a subject of the same age, and a subject with the same disorder, disease, or condition.

Together, the above described improvements may allow the subject to return to work and leisure activities more quickly and reduce hospital time and costs.

### EXAMPLES

### Example 1

### Subject Eligible for Microdiskectomy

A subject who is suffering from low back pain and R leg pain in the distribution of the L4 NR is seen by his general practitioner (GP), who recommends conservative treatment (e.g., rest and analgesics) for a period of 6 weeks. After 6 weeks, the subject returns to the GP complaining that the pain has not resolved. The subject is referred to a spine interventionalist to determine if the subject should undergo a partial or full diskectomy. After evaluating the patient, including a physical exam documenting radiculopathy in an L4 distribution, a positive straight leg raising test, and MRI assessment, the spine interventionalist determines that the patient has a herniated disk at L4 and is eligible for a microdiskectomy based on the subject meeting generally accepted guidelines for such a procedure, including MRI findings of HD at the appropriate level, the persistent pain of the subject for more than 6 weeks, and the failure of conventional conservative treatment. The spine interventionalist, based on the subject's eligibility for the partial diskectomy, recommends that the subject undergo an induction treatment regimen with a TAT, specifically a direct TNF-I, such as etanercept, adalimumab, or infliximab, or an NFκB-I in a depot formulation, to delay the need for the surgery or to improve the outcome of the surgery, should it ultimately result. The spine interventionalist administers etanercept, epidurally via a transforaminal approach injection under fluoroscopic guidance to the L4 nerve root in a total volume between 0.1 and 5 mls (see Table 1 for dose ranges illustrative of typical induction and maintenance regimens). Optionally, between 2 and 4 weeks later, the patient is reevaluated by the spine interventionalist, who decides to repeat the epidural injection via a translaminar, non-flouroscopic guided approach. The spine interventionalist administers a similar dose in a similar total volume, which may be optionally increased if given through a translaminar midline epidural approach.

The subject is then is referred back to his GP for follow up, and the GP prescribes a maintenance regimen of a parenterally administered TAT. The TAT, which may be a TNF-I such as etanercept, adalimumab, or infliximab, is administered by IV infusion, or intramuscular, or SC injection at intervals, for example, weekly or bi-weekly, up to monthly, or less frequently, for a period of from 2 months to 10 years. The subject is assessed post-administration using objective and subjective assessment tools, including one or more of the following: the Roland disability questionnaire, the Oswetry disability questionnaire, the VAS pain scale, the Likert scale, an MRI evaluation, and a neurological assessment.

### Example 2

### Subject Eligible for Facet Joint Replacement

A subject suffering from right sided back and buttock pain is seen by his GP, who recommends conservative treatment (e.g., rest, analgesics) for a period of 8 weeks. During this period, the subject independently attends a chiropractic practitioner, but experiences no lasting relief from the chiropractor's manipulations. After 8 weeks, the subject returns to the GP complaining that the pain has not resolved. The GP, therefore, refers the subject to a spine surgeon, who evaluates the subject.

The spine surgeon performs a physical evaluation of the subject, which includes demonstrating a reproduction of pain with ipsilateral bending, mild point tenderness over the affected facet joint, and a consistent distribution of the subject's pain over a lumbar facet innervation pattern. Based on these observations, the spine surgeon determines that the patient has severe facet arthropathy in the R L3-4 facet joint, based on the subject meeting the generally accepted guidelines for such a diagnosis, including physical exam, possible x-rays or MRI showing significant facet arthritic changes, and the failure of conventional conservative treatment. The pain physician informs the subject that he is eligible for a facet joint replacement, and recommends that he first undergo a course of induction treatment with an intrafacet joint injection of a TAT, specifically a TNF-I such as etanercept, to potentially forestall or eliminate the need for the facet joint replacement. Under fluoroscopic guidance, the spine surgeon administers the direct TNF-I in a total volume of between 0.1 and 2 ml, into the affected facet joint in the subject. Since the pain is sometimes referred from the adjacent facet, the two adjacent facets on the right side, at L2-L3 and L4-L5 are treated in a similar manner at the same juncture with intrafacet joint injections of the TNF-I. The subject is then referred back to his GP for follow up.

The GP begins a maintenance program of an IV TAT, in which the TAT, which may be a direct TNF-I, or may be another TAT, is administered at intervals, for example, from as frequently as once a month to once each 6 months, for a period of from 2 months to 10 years. The subject is assessed post-administration using objective and subjective assessment tools, including one or more of the following: the Roland disability questionnaire, the Oswetry disability questionnaire, the VAS pain scale, the Likert scale, and a neurological assessment.

### Example 3

### Subject with Complex Regional Pain Syndrome Type I

A female subject with a stressful home life notes severe pain in her right leg after sitting crossed legged on the floor in a position similar to the ‘Lotus position’ for an extended period of time. The subject attends school, however, but has to limp, and notices pain when her clothing touches the skin of her leg. Over the next two days the subject's pain increases, and her leg begins to look slightly red and swollen. The subject sees her pediatrician, who initially examines the subject for signs of bug bites in the days prior to the start of her pain. However, the pediatrician does not observe any hallmark signs of bites, scratches, or breaks in the subject's skin. The pediatrician recommends that the girl does not attend school for a few days, rests at home, and takes a mild oral analgesic if the pain continues. Over the next few days, however, the subject's leg continues to worsen, becoming increasingly swollen with a shiny appearance, and the subject can no longer wear any clothing on her leg due to extreme sensitivity to touch. Furthermore, the subject fails to feel any beneficial effect from treatment with the pediatrician-recommended analgesics. Due to increasing parental concern, the subject returns to her pediatrician, who refers the girl to an orthopedic physician.

The orthopedic physician recognizes Complex Regional Pain Syndrome (CRPS) Type I, and refers the subject to a pain specialist, who agrees with the diagnosis of CRPS Type I due to the findings of hyperalgesia, the neuropathic qualities of the pain described, and the appearance of the limb, and recommends an IV regional perfusion technique with a TAT. The subject is treated as an outpatient, and has a small IV catheter inserted into her right ankle/foot area. An arterial tourniquet is then applied mid thigh in an area of decreased hyperalgesia. An elastic rubber tourniquet is then wrapped, from distal to proximal, so as to exsanguinate the limb of venous blood. Once the limb is exsanguinated, the arterial tourniquet is inflated to a pressure of 20 mmHg above systolic. The elastic tourniquet is then removed and the TAT is injected through the previously placed IV catheter in a total volume of between 10 and 100 ml. The limb is then allowed to remain ischemic for a period of 30 minutes, or until the subject's pain associated with the limb tourniquet becomes too severe for her to tolerate, or whichever comes first. The tourniquet is then slowly deflated, the IV catheter removed, and the subject discharged home. This entire procedure is repeated from 0 to 3 times, at weekly to every two week intervals. The subject is then referred back to her pediatrician for follow up.

The pediatrician begins a maintenance program of a SC TAT, in which a TAT, which may be a direct TNF-I, or may be another TAT, is administered at intervals, potentially from as frequently as once a month to once each 6 months, for a period of from 2 months to 10 years. The subject is assessed post-administration using objective and subjective assessment tools, including one or more of the following: the Roland disability questionnaire, the Oswetry disability questionnaire, the VAS pain scale, the Likert scale, and a neurological assessment.

### Example 4

### Subject with Post-Operative Pain

A 65 year old moderately obese subject has noticed increased pain when walking in her right hip joint. The subject is seen by her GP, who notes that the pain pattern is consistent with hip pain, and that it can be reproduced by direct compression of the right leg into the hip joint. The GP refers the subject to an orthopedic surgeon who obtains hip films showing complete degeneration of the joint space including the femoral head and acetabulum. Based on these observations, the orthopedic surgeon recommends hip replacement. The surgeon also informs the subject that recovery times in such procedures are typically limited by the pain associated with inflammation surrounding the new prosthesis and joint. The surgeon further advises that this is very likely to be an issue in her case, due to her relatively sedentary lifestyle, and recommends that the subject undergo an induction treatment regimen with a TAT, for example a direct TNF-I, which will be administered, for example, during surgery. The subject consents, and the surgeon performs the operation, inserting a bipolar prosthesis with a femoral and acetabular component. Before closing the wound, the surgeon directly injects an induction regimen solution containing a TAT, for example, etanercept, in a total volume of between approximately 2 and 50 ml, into the pericapsular space, surrounding muscles, and directly intracapsularly.

During the 6-month recuperative period following surgery and in the following months and years, the subject visits the orthopedic surgeon as an outpatient at intervals of, for example, one week, one month, two months, and 6 months. During these visits, the orthopedic surgeon administers a maintenance program of a TAT, in which a TAT, which may be a direct TNF-I such as infliximab, or may be another TAT, is administered IV, SC or intramuscularly, at intervals, potentially from as frequently as once a week or once a month, to bimonthly, to once every 6 months, for a period of from 2 months to 10 years. As required, the subject is assessed using objective and subjective assessment tools, including one or more of the following: the Roland disability questionnaire, the Oswetry disability questionnaire, the VAS pain scale, the Likert scale, and a neurological assessment.

### Example 5

### Subject with Pure Diskogenic Pain without Radicular Component

A subject suffering from low back pain is seen by his GP, who recommends conservative treatment (e.g., rest and analgesics) for a period of 6 weeks. After 6 weeks, the subject returns to the GP, complaining that the pain has not resolved. After a period of 6 months, the subject is finally referred by the GP to a spine interventionalist.

The spine interventionalist examines the subject and determines from the midline distribution of pain in the lower lumbar spine, and by having the subject perform forward and backward bending tests, that the subject might suffer be suffering from diskogenic pain. Provocative diskography is scheduled, and the subject's diskogenic pain symptoms are reproduced upon injection of fluid into the L3-4 disk. To further confirm the diagnosis, the subject is given a functional anesthetic diskography temporarily implantable pump, manufactured by Kyphon, with a catheter remaining in the affected disk for about two weeks. During the next two weeks, upon experiencing the pain, the subject is administered either a local anesthetic, a short-acting TAT such as a p38I, or a saline solution. The p38I and the LA completely mask the patient's symptoms, while the saline does not affect the symptoms. Though the subject would thus be eligible for a diskectomy or intradiskal electrothermal therapy (IDET), the spine interventionalist injects a small volume of an induction dose of a long-acting TAT, specifically a microsphere formulation of a direct TNF-I, at this time into the L3-4 disk in order to initiate therapeutic treatment early and potentially avoid a more invasive treatment option. By monitoring the subject's response, the spine surgeon determines whether a second intradiskal injection of the induction dose might follow the initial induction dose following a period of one to seven days, 2 weeks, four weeks, and up to 6 months. Following the end of the induction regimen, the subject is referred back to his GP for follow up.

The GP begins a maintenance program of perispinal intramuscular, bilateral injections of the TAT, in which a TAT which may be a direct TNF-I such as infliximab, or may be another TAT, is administered at intervals, potentially from as frequently as once a month to once each 6 months, for a period of from 2 months to 10 years. The subject is assessed post-administration using objective and subjective assessment tools, including one or more of the following: the Roland disability questionnaire, the Oswetry disability questionnaire, the VAS pain scale, the Likert scale, and a neurological assessment.

### Example 6

### Subject with Lumbago (Non-Specific Lower Back Pain of Non-Radicular, Non-Zygopophyseal, Non-Diskogenic Origin)

A subject suffering from low back pain is seen by his GP, who recommends conservative treatment (e.g., rest, analgesics) for a period of 6 weeks. After 6 weeks, the subject returns to the GP, complaining that the pain has not resolved. The subject is referred to a pain specialist, who examines the patient and determines from the distribution of pain in the lower back, and negative tests for disk, facet, or radicular signs, that the patient is suffering from back pain, not otherwise specified, in the lumbar region. The pain specialist thus injects a volume of an induction dose of a TAT, specifically a depot formulation of an NFκB-I or a direct TNF-I such as etanercept, into the epidural space at this time in the L3-4 interspace via a midline approach, and injects a total volume of between 1 and 30 ml of the TAT. By monitoring the subject's response, the pain specialist determines whether a second epidural injection of the TAT in a similar manner and volume of the induction dose might follow the initial induction dose following a period of one to seven days, 2 weeks, four weeks, and up to 6 months. The subject is then referred back to his GP for follow up.

The GP begins a maintenance program of perispinal intramuscular, bilateral injections of the TAT, in which a TAT, which may be a direct TNF-I, or may be another TAT, is administered at a regular interval by IV, intramuscular, or SC administration, potentially from as frequently as once a month to once each 6 months, for a period of from 2 months to 10 years. The subject is assessed post-administration using objective and subjective assessment tools, including one or more of the following: the Roland disability questionnaire, the Oswetry disability questionnaire, the VAS pain scale, the Likert scale, and a neurological assessment.

### Example 7

### Subject at Risk for Post-Operative Ileus

A 50 year old male subject notices blood in his stools. He consults his GP who performs a guiaic test and confirms the presence of occult blood. The GP then refers the subject to a gastroenterologist, who performs a colonoscopy and diagnoses a colon cancer in the transverse colon. The patient is next referred to a general surgeon who performs an open laparotomy and primary resection of the tumor. Because the bowel is heavily manipulated during the surgery, the subject is at substantial risk for development of post operative ileus, which can delay discharge and lead to substantial pain and morbidity. The surgeon is well aware that manipulation of the bowel in this manner typically results in an intense localized inflammatory response, including a marked neutrophil infiltration into the bowel, that is the histologic hallmark of the inflammation accompanying post operative ileus. To reduce the risk of post-operative ileus, therefore, the surgeon sprays the bowel with an induction dose of a TAT, a direct TNF-I such as etanercept, prior to closing the peritoneum. During the subject's post-operative recovery in the hospital, the surgeon prescribes a maintenance regimen of a TAT, which may be a direct TNF-I or another TAT, in order to continue the anti-inflammatory effect achieved by the locally administered TAT induction regimen. The maintenance regimen includes 1 to 4, or more, doses of TAT by SC or intramuscular administration.

### Example 8

### Subject with Rheumatoid Arthritis

A 45 year old subject starts to feel unwell over the course of several months and notices symptoms of fatigue, lack of appetite, low grade fever, muscle and joint aches, and stiffness. The subject's muscle and joint stiffness is usually most notable in her wrists and hands and in the morning and after periods of inactivity. The subject starts taking ibuprofen (e.g., Motrin® and Advil®) to treat her symptoms. The subject also notes her joints frequently become red, swollen, painful, and tender, at periods when her pain is particularly severe. The subject eventually seeks care from a GP, who prescribes a course of corticosteroids. The GP also notices the symmetric distribution of joint involvement, which she recognizes as potential indicia of rheumatoid arthritis, and sends her to a rheumatologist.

The rheumatologist performs blood tests, which show positive rheumatoid factor and an elevated sedimentation rate; he also obtains x-rays of her hands and wrists that show minimal arthritic changes, but synovial thickening that is consistent with rheumatoid inflammation. Due to her inability at this time to complete activities of daily living without severe pain in her hands and wrists, the rheumatologist sends the subject to a hand surgeon, who injects small volumes of an induction dose of a TAT, specifically a direct TNF-I such as adalimumab, into her wrist joint and several carpal-metacarpal joints that are particularly severely affected. The rheumatologist continues a chronic maintenance regimen of TAT, which may be a direct TNF-I such as etanercept, or another TAT, administered either subcutaneously, intramuscularly, or intravenously.

### Example 9

### Subject with Alzheimer's Disease

A 78 year old subject is observed by his wife to be increasingly forgetful. At first the subject forgets only minor things; however, he gradually becomes more forgetful and begins to forget details to events and procedures that he has known his whole life. He consults his GP, who refers him to a neurologist to be evaluated for possible Alzheimer's disease.

The neurologist conducts a careful physical exam to rule out other possible organic causes of dementia, and orders a battery of assessments, including a magnetic resonance imaging (MRI) scan, a Positron emission tomography (PET) scan, and neuropsychological testing. The tests reveal a pattern of brain atrophy with decreased activity on PET scan, which are results known to be consistent with early to moderate Alzheimer's disease. In order to mitigate the progression of the disease, the neurologist performs an intrathecal injection with a small volume of an induction dose of a TAT, specifically a direct TNF-I. This is repeated at two to four week intervals for a period of four months. The subject is scheduled for epidural maintenance injections of the TAT, which may be a TNF-I, or may be another TAT, every three months. Follow up assessments are performed periodically using PET, single photon computed tomography (SPECT), MRI and other appropriate imaging procedures in order to monitor the subject's progress.

### Example 10

### Subject with Type II Diabetes

A 64 year old subject notices that he is always thirsty and needs to urinate more frequently. The subject also notices that he feels occasional flu-like symptoms, has fluctuating weight, is having increased problems with his vision, has minor wounds and cuts that tend to heal more slowly, has sore gums, and experiences an occasional tingling paresthesia in his legs.

The subject sees his GP, who recognizes a pattern of diabetes and orders fasting blood sugar, glucose tolerance, and hemoglobin AIC tests, which confirm the diagnosis of diabetes. The GP then refers the subject to a gastroenterologist for a new treatment that may lessen the subject's hypoinsulinemia and improve pancreatic islet cell production of insulin.

The gastroenterologist informs the subject that the new treatment involves a novel regimen involving a TAT administered as part of an induction and maintenance regimen. The gastroenterologist also advises the subject that in his case the induction regimen will involve injection of a TAT, for example, directly into the pancreatic duct. The subject consents and the gastroenterologist performs endoscopic retrograde cholecystopancreatography [or endoscopic retrograde cholangiopancreatography] (ERCP), during which an induction dose of the TAT, specifically a direct TNF-I, is injected directly into the pancreatic duct. Following this procedure, the subject is seen by an endocrinologist. In addition to standard anti-diabetic drug therapy, the endocrinologist prescribes a maintenance regimen of a TAT, which may be a direct TNF-I, or may be another TAT. The maintenance regimen is administered every two to eight weeks via SC or intramuscular injection performed either by the subject, or by a health professional such as the subject's primary care physician or nurse practitioner. The subject is followed in a manner similar to that used for all Type II diabetic subjects, for example, those not treated with a TAT. The subject's TAT maintenance regimen is continued, and the dose is periodically adjusted based upon further testing.

### Example 11

### Subject with Phantom Limb Pain

A 27 year old soldier suffers a severe injury to his right leg while serving overseas. The extent of the soldier's injury is so severe that his right leg has to be amputated above the knee. In the week following surgery, the soldier undergoes treatment with IV calcitonin, but does not experience any relief from the extent, frequency and severity of the discomfort caused by his surgery. The soldier is then returned home to the United States. Shortly after arriving back in the U.S., the soldier experiences sensations of burning and twisting in his missing limb and toes. The soldier notes that his pain is triggered when he uses his prosthesis with any pressure, when he is emotionally wrought or tired, and by certain climate changes. The soldier's army neurologist examines the subject and prescribes gabapentin and desipramine for his symptoms. The soldier notes a moderating effect with this treatment, but complains that his symptoms are still debilitating. Eight weeks later, the soldier's army neurologist examines his healed limb stump and observes that pinpoint pressure in three areas of the stump cause the soldier to feel severe pain and discomfort, and suspects neuroma. The army neurologist elects to treat the soldier with an induction regimen of a direct TNF-I such as etanercept, by direct injection into the areas of presumed neuroma, using a total volume between 1 and 10 ml into each area, containing doses as noted in FIGS. 3-5. The army neurologist sees the soldier again three weeks later and repeats the TNF-I injections at locations that remain capable of reproducing the symptoms the soldier is experiencing. The army neurologist then refers the soldier back to the care of the medical internist, who initiates a maintenance regimen of intramuscular injections of a direct TNF-I, such as etanercept, given once per month, for several months, and then once every 6 months continuously.

### REFERENCES

1 Armour K L et al. (1999). Recombinant human IgG molecules lacking Fcgamma receptor I binding and monocyte triggering activities. Eur. J. Immunol. 29:2613-2624.
2 De AU and Pal D (1975). Possible antineoplastic agents I. J. Pharm. Sci. 64(2):262-266.
3 List A et al. (2005). Efficacy of Lenalidomide in Myelodysplastic Syndromes. N. Eng. J. Med. 352:549-57.
4 Muto M (2007). Intradiskal and Intraforaminal Oxygen-ozone Therapy in the Treatment of Herniated Disks. Abstract presented at the American Society of Spine Radiology Annual Symposium, February 22-25 in Marco Island, Fla.
5 Katz J and Melzack R (1999). Measurement of Pain. Surg Clin North Am. 79(2):231-52.
6 Caraceni A et al. (2002). Pain measurement tools and methods in clinical research in palliative care: recommendations of an Expert Working Group of the European Association of Palliative Care. J Pain Symptom Manage 23(3):239-55.

What is claimed is:

1. A method for treating a disease or disorder, comprising administering to a subject in need thereof an induction regimen of a direct TNF inhibitor (direct TNF-I) and a maintenance regimen of a direct TNF-I, wherein the TNF-I of the induction regimen is administered at a lower dose per administration than the dose per administration of the TNF-I of the maintenance regimen, and wherein the TNF-I of the induction regimen is administered more locally and/or in a more invasive manner than the TNF-I of the maintenance regimen.

2. A method for treating a disease or disorder, comprising administering to a subject in need thereof an induction regimen of an NFκB inhibitor (NFκB-I), and a maintenance regimen of an NFκB-I, wherein the NFκB-I of the induction regimen is administered at a lower dose per administration than the dose per administration of the NFκB-I of the maintenance regimen, and wherein the NFκB-I of the induction regimen is administered more locally and/or in a more invasive manner than the NFκB-I of the maintenance regimen.

3. The method of claim 1 or 2, wherein the disease or disorder comprises a pain syndrome, a spinal disorder, an orthopedic disorder, an inflammatory disease, an immune system disease, a metabolic disorder, a cardiovascular disease, a disease of endothelial dysfunction, a disorder of the central nervous system, and a disorder of the peripheral nervous system.

4. The method of claim 3, wherein the pain syndrome is selected from the group consisting of acute pain, chronic pain, complex regional pain syndrome type I, complex regional pain syndrome type II, neuropathic pain, post-operative pain, pain caused by inflammation, chronic lower back pain, sciatica, cluster headaches, post-herpetic neuralgia, phantom limb pain, stump pain, central pain, dental pain, opioid-resistant pain, visceral pain, surgical pain, bone injury pain, pain during labor and delivery, pain resulting from burn, post partum pain, migraine, angina pain, fibromyalgia, and genitourinary tract-related pain, including cystitis, and nociceptive pain.

5. The method of claim 3, wherein the spinal disorder is selected from the group consisting of disk disorders including herniated disk (HD) and DDD, disorders of spinal stability, disorders of the vertebrae including kyphosis and facet joint disease, nerve disorders, SS, arthritic spinal disorders, back pain conditions, and failed back surgery syndrome.

6. The method of claim 5, wherein the disk disorder is a herniated disk or a degenerative disk disorder.

7. The method of claim 6, wherein the disk disorder is selected from the group consisting of prolapsed disk, protruding disk, extruded disk, bulging disk, sequestered disk, DDD, DDD with internal disk derangement, diskogenic pain, annular disorder, annular bulge, annular tear, nucleus pulposus degeneration, nerve root (NR) compression, radicular pain, radiculopathy, sciatica, radiating pain, and distraction injury.

8. The method of claim 5, wherein the disorder of spinal stability is selected from the group consisting of spondylolysis, spondylolisthesis, lytic spondylolisthesis, degenerative spondylolisthesis, lumbar spondylolisthesis, isthmic spondylolisthesis, and grade 1 spondylolisthesis.

9. The method of claim 5, wherein the vertebral disorder is selected from the group consisting of vertebral body collapse, vertebral body degeneration, vertebral body compression, metastasis, kyphosis, facet joint disease, facet disease, facet joint disease facet joint syndrome, and impinging facet joints.

10. The method of claim 5, wherein the arthritic spinal disorder is selected from the group consisting of rheumatoid arthritis, ankylosing spondylitis, osteoarthritis, degenerative spinal arthritis, cervical arthritis, thoracic arthritis, DDD, bone spurs, osteophytes, and an arthritic facet joint disorder.

11. The method of claim 5, wherein the nerve disorder is selected from the group consisting of nerve compression syndrome, nerve root (NR) compression, NR irritation, NR inflammation, nerve entrapment, nerve compression by a tumor, lumbago, HD, SS, neural foraminal narrowing, pinched nerve, and sciatica.

12. The method of claim 5, wherein the back pain condition is selected from the group consisting of back pain, low back pain, chronic back pain, radicular pain, radiating pain, sciatica, radiculitis, lumbar radiculopathy, diskogenic pain, facet pain, cervical radiculopathy, cervical headache, whiplash, whiplash headache, whiplash associated disorder, scoliosis, scoliosis pain, post-operative pain, post-operative leg pain, and fibromyalgia.

13. The method of claim 3, wherein the orthopedic disorder is selected from the group consisting of an orthopedic joint disorder of the hip, knee, shoulder, ankle, elbow, wrist, toe, finger, sacro-iliac, and spinal facet joint.

14. The method of claim 3, wherein the inflammatory disorder is selected from the group consisting of chronic inflammatory airway disorders (including asthma, alergic asthma, non-allergic, intrinsic asthma, exercise-induced asthma, nocturnal asthma, occupational asthma, steroid resistant asthma, exercise-induced bronchospasm, and chronic obstructive pulmonary disease); chronic inflammatory bowel diseases (including ulcerative colitis, and Crohn's disease); chronic inflammatory connective tissue diseases (including lupus erythematosus, scleroderma, Sjogren's syndrome, poly- and dermatomyositis, vasculitis, and MCTD); chronic inflammatory joint diseases (including rheumatoid arthritis juvenile chronic arthritis, Still's disease, rheumatoid spondylitis, lupus erythematosus, ankylosing spondylitis, psoriatic arthritis, and reactive arthritis, rheumatoid arthritis of the hip, bursitis of the hip, and osteoarthritis of the hip); chronic inflammatory skin diseases (including psoriasis, diskoid lupus erythematosus, scleroderma, hives, rosacea, dermatitis, and atopic dermatitis); spondyloarthropies; cardiomyopathy; atherosclerosis vasculitis (including anti-neutrophil cytoplasmic Ab (ANCA)-associated vasculitis and chronic and relapsing ANCA-associated vasculitis); acute renal disease; chronic renal disease; glomerulonephritis; inflammatory eye disorders (including retinitis); tuberculosis; chronic cholecystitis; bronchiectasis; Hashimoto's thyroidiitis; Silicosi; pneumoconioses; hyper-IgG4 disease; ileus; inflammatory side effects associated with a pharmaceutical agent; and post operative inflammation.

15. The method of claim 1 or 2, wherein the treatment is administered peri-operatively to a surgery of the subject.

16. The method of claim 15, wherein the surgery is an orthopedic surgery selected from the group consisting of surgery to the hand, elbow, shoulder, spine, hip, knee, or ankle joint, arthroscopy (including of the wrist, elbow, shoulder, spine, hip, knee, ankle, or any other joint); carpal tunnel release; knee arthroscopy (including with meniscectomy, chondroplasty or anterior cruciate ligament reconstruction); tendon repair or replacement (including rotator cuff tendon repair); ligament repair or replacement; fracture repair; and bone graft.

17. The method of claim 15, wherein the surgery involves the implantation revision, or removal of an orthopedic device used for replacement or repair of a joint structure of the hand, foot, wrist, elbow, shoulder, spine, hip, knee, or ankle joint.

18. The method of claim 17, wherein the device is selected from the group consisting of a stent; a pump; an annular repair device; a nucleus replacement device; a dynamic stabilization device; a synthetic bone graft substitute; an allograft cage; a motion preservation device; a pedicle screw; a facet screw; a vertebral body replacement; a hip replacement device; a knee replacement device; a shoulder replacement device; a wrist replacement device; an ankle replacement device; and an inter-vertebral disk replacement device (artificial disk device).

19. The method of claim 1 or 2, wherein the induction regimen is administered locally to a site of pain.

20. The method of claim 1 or 2, wherein the induction regimen is administered locally to a site of inflammation.

21. The method of claim 1 or 2, wherein the induction regimen is administered locally to an organ.

22. The method of claim 1 or 2, wherein the induction regimen is administered locally to a joint.

23. The method of claim 1 or 2, wherein the induction regimen is administered locally to the spine.

24. The method of claim 1 or 2, wherein the induction regimen route of administration is selected from:
intra-operative, intracerebral, intracerebroventricular, into an organ selected from intracardiac, intraventricular, and intracoronary administration; endoscopic retrograde cholangiopancreatography; intrapleural, intraperitoneal, intradiskal administration; intra-articular or intracapsular administration; peridiskal administration; pericapsular administration; intramedullary administration; intrathecal administration; epidural administration (including periradicular and transforaminal administration); intra-facet administration; intra-cartilaginous administration; and epidural, intrapleural, or intraperitoneal administration, and
the maintenance regimen route of administration is selected from:
IV, perispinal, intramuscular, SC, oral, intranasal, buccal; inhalation (including intrapulmonary and intrabronchial); and transdermal administration.

25. The method of claim 5, wherein the induction regimen is administered locally to a site in or adjacent to one or more intervertebral disks, in or adjacent to one or more vertebra(e), or adjacent to one or more spinal nerve root(s) or nerve(s).

26. The method of claim 5, wherein the induction regimen is administered using intra-operative administration.

27. The method of claim 26, wherein the intra-operative administration includes administration into or adjacent to one or more spinal structure(s) selected from spinal NR(s) or nerve(s), intervertebral disk(s), vertebra(e), and dura.

28. The method of claim 5, wherein the maintenance regimen is administered using IV, perispinal, intramuscular, SC, oral, intranasal, buccal; inhalation (including intrapulmonary and intrabronchial); and transdermal administration.

29. The method of claim 28, wherein the maintenance regimen is administered using a catheter and a pump.

30. The method of claim 28, wherein the maintenance regimen is administered by implantation of a depot formulation, controlled-release, or hydrogel formulation.

31. The method of claim 5, wherein the induction regimen is administered using intradiskal, peridiskal, epidural (including periradicular and transforaminal), intradiskal/peridiskal, intradiskal/epidural, intradiskal/peridiskal/epidural or intra-facet administration, and the maintenance route of administration is selected from; IV, perispinal, intramuscular, SC, oral, intranasal, buccal; inhalation (including intrapulmonary and intrabronchial); and transdermal administration.

32. The method of claim 31, wherein the induction regimen comprises implantation of a depot formulation, controlled-release, or hydrogel formulation.

33. The method of claim 31, wherein the maintenance regimen comprises implantation of a depot formulation, controlled-release, or hydrogel formulation.

34. The method of claim 31, wherein the induction regimen is administered using epidural administration and the maintenance route of administration is selected from: IV, perispinal, intramuscular, SC, oral, intranasal, buccal, inhalation (including intrapulmonary and intrabronchial); and transdermal administration.

35. The method of claim 34, wherein the induction regimen comprises implantation of a depot formulation, controlled-release, or hydrogel formulation.

36. The method of claim 34, wherein the maintenance regimen comprises implantation of a depot formulation, controlled-release, or hydrogel formulation.

37. The method of claim 5, wherein the induction regimen is completed prior to beginning administration of the maintenance regimen.

38. The method of claim 5, wherein the maintenance regimen begins at or near the same time as the induction regimen.

39. The method of claim 5, wherein the induction regimen direct TNF-I and the maintenance regimen direct TNF-I are the same.

40. The method of claim 5, wherein the induction regimen NFκB-I and the maintenance regimen NFκB-I are different.

41. The method of claim 5, wherein the direct TNF-I is selected from the group consisting of an antibody or antibody fragment, a fusion protein, a peptide, a SMIP, a small molecule, an oligonucleotide, an oligosaccharide, a soluble cytokine receptor or fragment thereof, a soluble TNF receptor Type I or a functional fragment thereof, a polypeptide that binds to TNF, and a dominant negative TNF molecule.

42. The method of claim 41, wherein the oligonucleotide is an siRNA.

43. The method of claim 41, wherein the direct TNF-I is selected from the group consisting of is selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP).

44. The method of claim 5, wherein the NFκB-I is selected from the group consisting of sulfasalazine, sulindac, clonidine, helenalin, wedelolactone, pyrollidinedithiocarbamate (PDTC), IKK-2 inhibitors, and IKK inhibitors.

45. The method of claim 1 or 2, further comprising administering to the subject a therapeutically effective amount of a supplemental active ingredient (SAI).

46. The method of claim 45, wherein the SAI is selected from the group consisting of a second TAT, a corticosteroid, ozone, an antirheumatic drug, a local anesthetic, a neuroprotective agent, a salicylic acid acetate, a hydromorphone, a non-steroidal anti-inflammatory drug, a cox-2 inhibitor, an antidepressant, an anticonvulsant, a calcium channel blocker, and an antibiotic.

47. A kit, comprising;
a) at least one container comprising an induction regimen of a direct TNF-I or an NFκB-I;
b) a delivery vehicle to administer the induction regimen of a direct TNF-I or an NFκB-I;
c) instructions for administration of the induction regimen of a direct TNF-I or an NFκB-I.

48. The kit of claim 47, wherein the delivery vehicle is selected from the group consisting of a syringe, a needle, a catheter, or a pump.

49. The kit of claim 47, further comprising at least one SAI.

50. The kit of claim 47, wherein delivery vehicle is adapted for an induction regimen to be administered using intra-operative administration.

51. The kit of claim 47, wherein the delivery vehicle is adapted for an induction regimen to be administered using an intradiskal, peridiskal, or epidural (including periradicular and transforaminal) administration, or any combination thereof, or intra-facet administration.

52. A kit, comprising;
a) at least one container comprising an induction regimen of a direct TNF-I or an NFκB-I;
b) a delivery vehicle to administer the induction regimen of a direct TNF-I or an NFκB-I;
c) at least one container comprising a maintenance regimen of a direct TNF-I or an NFκB-I;
d) a delivery vehicle to administer the maintenance regimen of a direct TNF-I or an NFκB-I; and
e) instructions for administration of the induction regimen of a direct TNF-I or an NFκB-I and the maintenance regimen of a direct TNF-I or an NFκB-I.
